(19) United States
(12) Patent Application Publication
(10) Pub. No.: US 2009/0111786 A1
(43) Pub. Date: Apr. 30, 2009

(54) Compounds that Prevent Macrophage Apoptosis and Uses Thereof

(76) Inventors: Christopher K. Glass, San Diego, CA (US); Annabel E. Valledor, Begues (ES); Michael Karin, La Jolla, CA (US); Li-Chung Hsu, Taiwan (CN)

Correspondence Address: Medlen & Carroll, 101 Howard Street, Suite 350, San Francisco, CA, 94105, US

(21) Appl. No.: 11/792,154

(22) PCT Filed: Dec. 2, 2005
(86) PCT No.: PCT/US2005/043616

§ 371 Date: May 19, 2008

Related U.S. Application Data
(60) Provisional application No. 60/632,905, filed on Dec. 3, 2004.

(51) Int. Cl. A61K 31/58; C12N 5/00; A61K 31/19; A61P 31/04

(52) U.S. Cl. 514/172; 435/375; 514/557

(57) ABSTRACT

The present invention relates to microbial infection, and in particular, the reduction of apoptosis associated with microbial infection, the screening of Liver X Receptor agonist and/or Retinoid X Receptor agonist that reduce apoptosis, and the treatment and analysis of microbial infection in vivo. In one embodiment, the present invention relates to Liver X Receptor agonist and/or Retinoid X Receptor agonist including but not limited to an agonist increasing the activity of Liver X Receptor and/or Retinoid X Receptor.

This application claims priority to U.S. Patent Application No. 60/632,905, filed on Dec. 3, 2004.

This invention was made, in part, with government support under grant numbers ES10337, AI061712, DK063491 and HL56989 awarded by the National Institutes of Health. As such, the U.S. government has certain rights in the invention.

### FIELD OF THE INVENTION

The present invention relates to microbial infection, and in particular, the reduction of apoptosis associated with microbial infection, the screening of Liver X Receptor agonist and/or Retinoid X Receptor agonist that reduce apoptosis, and the treatment and analysis of microbial infection in vivo. In one embodiment, the present invention relates to Liver X Receptor agonist and/or Retinoid X Receptor agonist including but not limited to an agonist increasing the activity of Liver X Receptor and/or Retinoid X Receptor.

### BACKGROUND

Current treatments for bacterial infections rely upon antibiotics. However, published reports indicate that although antibiotics were initially miracle cures, they are now increasingly ineffective due to the emergence of new bacteria strains including many resistant “superbugs.” Compounding the superbug phenomena is the observation that as quickly as new antibiotics are used, the pathogenic bacteria populations shift towards refractory strains. Furthermore, antibiotics are minimally if not contra-indicated in patients with co-existing viral infections. Antibiotic treatment in such patients, while potentially effective against the bacteria, may potentiate the viral infection.

Thus, there is a need to find new ways to identify drugs that will reduce bacteria infections, and in particular bacteria infections within patients with viral infections.

### SUMMARY OF THE INVENTION

The present invention relates to microbial infection, and in particular, the reduction of apoptosis associated with microbial infection, the screening of Liver X Receptor agonist and/or Retinoid X Receptor agonist that reduce apoptosis, and the treatment and analysis of microbial infection in vivo. In one embodiment, the present invention relates to Liver X Receptor agonist and/or Retinoid X Receptor agonist including but not limited to an agonist increasing the activity of Liver X Receptor and/or Retinoid X Receptor.

In one embodiment, the present invention relates to the use of Liver X Receptor and Retinoid X agonists that increase the activity of Liver X Receptor and/or Retinoid X Receptor. In one embodiment, the present invention contemplates methods for identifying agents for reducing apoptosis of macrophage cells, particularly bacteria-induced apoptosis mediated by a Liver X Receptor and/or a Retinoid X Receptor. Such methods serve to distinguish agents that are drug candidates (agent) as anti-microbials. Certain embodiments of the method are designed to access the apoptosis reduction potential of agents by virtue of their in vitro and in vivo ability to reduce expression of proteins associated with apoptosis, apoptotic pathways and apoptotic death.

In one embodiment, the invention provides a method of modulating apoptosis in a cell, the method comprising administering an agent to a cell, wherein the cell comprises a Liver X Receptor and wherein administration increases Liver X Receptor activity such that apoptosis is modulated. In another embodiment, apoptosis is decreased. The present invention is not limited to any particular type of agent. Indeed, a variety of agents is contemplated, for example, a Liver X Receptor agonist and/or a Retinoid X Receptor agonist. In another embodiment, the method comprises administering an agent to a cell, wherein the agent is chosen from one or more of a small molecule, a protein, a peptide, a peptidomimetic, and a nucleic acid molecule. In another embodiment, the method comprises administering an agent to a cell, wherein the agent is chosen from one or more of a 24(S),25-epoxycholesterol (EC), T1317, and GW3965. In another embodiment, the method comprises administering an agent to a cell, wherein the agent is a derivative of one or more of 24(S),25-epoxycholesterol, T1317, and GW3965. The present invention is not limited to the targeting of any particular kind of cell. Indeed a variety of cells can be targeted (for example, an immunocyte, a white blood cell, a macrophage etc.). In one embodiment, the method comprises administering an agent to a cell, wherein the cell is a white blood cell. In another embodiment, the method comprises administering an agent to a cell, wherein the cell is a macrophage cell.

In other embodiments, the invention provides methods of treating microbial infections in a cell, comprising, a) providing: i) a cell with one or more symptoms of a microbial infection, wherein the cell comprises a Liver X Receptor and/or a Retinoid X Receptor; and ii) a formulation comprising an agent, wherein the agent comprises a Liver X Receptor agonist and/or a Retinoid X Receptor agonist; and b) contacting the cell with the formulation for increasing Liver X Receptor and/or Retinoid X Receptor activity under conditions such that the one or more symptoms of a microbial infection are reduced. In one embodiment, the cell is in one or more of a population of cells, a tissue or a patient. In one embodiment, the patient is an animal (e.g., a human, a domestic animal, a livestock animal, an exotic animal, etc.). In some embodiments, the microbial infection comprises infectious bacteria. The present invention is not limited to any particular kind of bacterium. Indeed, treatment of a variety of bacteria is contemplated (for example, Bacillus species, Escherichia species, etc.). In another embodiment, the patient has a microbial infection associated with one or more symptoms of a pathogen infection. It is not meant to limit the type of pathogen. Indeed, a variety of pathogens is contemplated (for example, a bacterium, a virus, etc.). In another embodiment, the infection is a multiple infection. In a further embodiment, the multiple infections comprise a bacterial infection and a viral infection. In one embodiment, the bacterium is selected from a group comprising Bacillus species, Yersinia species, Salmonella species, Shigella species, Streptococcus species and Haemophilus species. In another embodiment, the patient has a microbial infection associated with one or more symptoms of a viral infection. In yet a further embodiment, the virus is selected from a group comprising Influenzavirus species. The present invention is not limited to any type of agent. Indeed, a variety of agents is contemplated (for example, an engineered agent, a synthesized agent, etc.). In another embodiment, the agent is chosen from one or more of a small molecule, a protein, a peptide, a peptidomimetic, a nucleic acid molecule, and the like. In another embodiment, the agent is chosen from one or more of 9-cis retinoic acid, 24(S),25-epoxycholesterol, T1317, and GW3965. In yet another embodiment, the agent is chosen from one or more of a derivative of 9-cis retinoic acid, 24(S),25-epoxycholesterol, T1317, and GW3965.

In one embodiment, the invention provides a method of treating a microbial infection in an animal, comprising, a) providing: i) an animal with one or more symptoms of a microbial infection; and ii) a formulation comprising an agent, wherein the agent further comprises a Liver X Receptor agonist and/or a Retinoid X Receptor agonist; and b) administering to the animal the formulation for increasing Liver X Receptor activity and/or Retinoid X Receptor activity under conditions such that the one or more symptoms of a microbial infection are reduced. In one embodiment, the patient is an animal (e.g., a human, a domestic animal, a livestock animal, an exotic animal, etc.). In another embodiment, the animal is chosen from one or more of a domestic animal and a livestock animal. In another embodiment, the patient is a human. In one embodiment, the patient is a mouse. In another embodiment, the agent is chosen from one or more of a small molecule, a protein, a peptide, a peptidomimetic, and a nucleic acid molecule. In another embodiment, the agent is chosen from one or more of 9-cis retinoic acid, 24(S),25-epoxycholesterol, T1317, and GW3965. In another embodiment, the agent is chosen from one or more of a derivative of 9-cis retinoic acid, 24(S),25-epoxycholesterol, T1317, and GW3965. In another embodiment, the microbial infection is caused by a bacterium. The present invention is not limited to any particular type of bacterium. Indeed, a variety of bacteria are contemplated, including, but not limited to gram-negative bacterium, gram-positive bacterium, etc., for example, pathogenic bacterium, including, but not limited to Bacillus species, Yersinia species, Salmonella species, Shigella species, Streptococcus species and Haemophilus species. In a further embodiment, the invention provides a method for reducing apoptosis of macrophage cells, wherein the bacterium is gram-negative. In yet another further embodiment, the invention provides a method for reducing apoptosis of macrophage cells, wherein the bacterium is gram-positive. In another embodiment, the bacterium is selected from a group comprising Bacillus species, Escherichia species, Yersinia species, Salmonella species, and Shigella species. In another embodiment, the invention provides a method for reducing apoptosis of macrophage cells, wherein the macrophage cells are contacted with a molecule chosen from one or more of 9-cis retinoic acid, 24(S),25-epoxycholesterol, T1317, and GW3965. In another embodiment, the agent is chosen from one or more of a derivative of 9-cis retinoic acid, 24(S),25-epoxycholesterol, T1317, and GW3965.

In one embodiment, the invention provides a method for modulating anti-apoptotic activity in a cell, comprising, a) providing: i) a cell with one or more symptoms of a microbial infection, wherein the cell comprises an anti-apoptotic gene; and ii) a formulation comprising a Liver X Receptor agonist and/or a Retinoid X Receptor agonist; and b) contacting the cell with the formulation under conditions such that an anti-apoptotic gene activity is increased in the cell. In another embodiment, the increase in an anti-apoptotic gene activity results in reduction of one or more symptoms of a microbial infection. In another embodiment, the microbial infection is caused by a bacterium. In another embodiment, the bacterium is selected from the group comprising Bacillus species, Escherichia species, Yersinia species, Salmonella species, and Shigella species. It is not meant to limit the type of anti-apoptotic gene. Indeed, a variety of anti-apoptotic genes is contemplated. In another embodiment, the anti-apoptotic gene is chosen from one or more of AIM, Birc1a, and Bcl-X_{L}. In another embodiment, the method further comprises contacting the cell with one or more of a small molecule, a protein, a peptide, a peptidomimetic, and a nucleic acid under conditions such that an anti-apoptotic gene activity is increased in the cell.

In one embodiment, the invention provides a method for modulating anti-apoptotic gene activity in a patient, comprising, a) providing: i) a patient with one or more symptoms of a microbial infection, wherein the patient comprises an anti-apoptotic gene; and ii) a formulation comprising a Liver X Receptor agonist and/or a Retinoid X Receptor agonist; and b) administering the formulation to the patient under conditions such that activity of an anti-apoptotic gene is increased in a patient. In another embodiment, the increase in anti-apoptotic gene activity is a reduction in one or more symptoms of a microbial infection. In another embodiment, the microbial infection is caused by a bacterium. In another embodiment, the bacterium is selected from a group comprising Bacillus species, Escherichia species, Yersinia species, Salmonella species, and Shigella species. In another embodiment, the anti-apoptotic gene is chosen from one or more of AIM, Birc1a, and Bcl-X_{L}. In another embodiment, the method comprises administering to the patient one or more of a small molecule, a protein, a peptide, a peptidomimetic, and a nucleic acid under conditions such that an anti-apoptotic gene activity is increased in a patient.

In one embodiment, the invention provides a method for modulating apoptotic gene activity in a cell, comprising, a) providing: i) a cell with one or more symptoms of a microbial infection, wherein the cell comprises an apoptotic gene; and ii) a formulation comprising a Liver X Receptor agonist and/or a Retinoid X Receptor agonist; and b) contacting the cell with the formulation under conditions such that activity of an apoptotic gene is decreased in the cell. In another embodiment, the decrease in apoptotic gene activity is the reduction of one or more symptoms of a microbial infection. In another embodiment, the microbial infection is caused by a bacterium. In another embodiment, the bacterium is selected from a group comprising Bacillus species, Escherichia species, Yersinia species, Salmonella species, and Shigella species. In another embodiment, the apoptotic gene is chosen from one or more of AIM, Birc1a, and BCl-X_{L}. In another embodiment, the method comprises delivering to the cell one or more of a small molecule, a protein, a peptide, a peptidomimetic, and a nucleic acid under conditions such that an apoptotic gene activity is decreased in the cell. It is not meant to limit the type of apoptotic gene. Indeed, a variety of genes is contemplated. In another embodiment, the apoptotic gene is chosen from one or more of Deoxyribonuclease I-like 3 (Dnase1L3), Caspase 1, Caspase 4, Caspase 11, Caspase 7, Caspase 12, Fas ligand, Cell death-inducing DFFA-like effector A (CIDE-A), and peptidoglycan recognition protein (Tag7). In another embodiment, the method further comprises contacting the cell with one or more of a small molecule, a protein, a peptide, a peptidomimetic, and a nucleic acid under conditions such that an apoptotic gene apoptotic gene activity is decreased in the cell.

In one embodiment, the invention provides a method for modulating apoptotic gene activity in a patient, comprising, a) providing: i) a patient with one or more symptoms of a microbial infection, wherein the patient comprises an apoptotic gene; and ii) a formulation comprising a Liver X Receptor agonist and/or a Retinoid X Receptor agonist; and b) administering the formulation to the patient under conditions such that an apoptotic gene activity is decreased in the patient. In another embodiment, the decrease in apoptotic gene activity is reduction of one or more symptoms of a microbial infection. In another embodiment, the microbial infection is caused by a bacterium. In another embodiment, the bacterium is selected from a group comprising Bacillus species, Escherichia species, Yersinia species, Salmonella species, and Shigella species. In another embodiment, the apoptotic gene is chosen from one or more of AIM, Birc1a, and Bcl-X_{L}. In another embodiment, the method further comprises administering to the patient one or more of a small molecule, a protein, a peptide, a peptidomimetic, and a nucleic acid under conditions such that an apoptotic gene activity is decreased in the patient. It is not meant to limit the type of apoptotic gene. Indeed, a variety of genes is contemplated. In another embodiment, the apoptotic gene is chosen from one or more of Deoxyribonuclease I-like 3 (Dnase1L3), Caspase 1, Caspase 4, Caspase 11, Caspase 7, Caspase 12, Fas ligand, Cell death-inducing DFFA-like effector A (CIDE-A), and peptidoglycan recognition protein (Tag7). In another embodiment, the method further comprises administering one or more of a small molecule, a protein, a peptide, a peptidomimetic, and a nucleic acid under conditions such that an apoptotic gene activity is decreased in the patient.

The present invention provides methods for modulating apoptosis, comprising administering an agent to a cell, wherein the cell comprises a liver X receptor (LXR) and wherein the administering increases activity of the LXR thereby modulating apoptosis. In some preferred embodiments, the modulating comprises reducing apoptosis. In some embodiments, the agent comprises one or more of a small molecule, a protein, a peptide, a peptidomimetic, and a nucleic acid. In some particularly preferred embodiments, the agent is an LXR agonist comprising one or more of a 24(S),25-epoxycholesterol (EC), T1317, and GW3965. In other preferred embodiments, the agent comprises an LXR agonist and a retinoid x receptor (RXR) agonist. Moreover, in some preferred embodiments, the cell is a myeloid cell, such as a macrophage.

Furthermore, the present invention provides methods of treating a microbial infection of a cell, comprising, providing: i) a cell with one or more symptoms of a microbial infection, wherein the cell comprises one or both of a liver X receptor (LXR) and a retinoid X receptor (RXR); and ii) a composition comprising an agent, wherein the agent comprises one or both of a LXR agonist and a RXR agonist; and contacting the cell with the composition under conditions suitable for increasing activity of one or both of LXR and RXR such that the one or more symptoms of the microbial infection are reduced. In some embodiments, the cell is in a population of cells, a tissue or an animal. In some preferred embodiments, the animal is a human or other mammal. In some particularly preferred embodiments, the microbial infection comprises a bacterial infection. In a subset of these embodiments, the bacterial infection comprises an infection with bacteria selected from the group consisting of Bacillus species, Escherichia species, Salmonella species, Shigella species, Yersinia species, Listeria species, Legionella species, Mycobacterium species, Streptococcus species and Haemophilus species. In some preferred embodiments, the agent comprises one or more of a 24(S),25-epoxycholesterol (EC), T1317, GW3965, and 9-cis-retinoic acid (9cRA). Moreover, in some preferred embodiments, the cell is a myeloid cell, such as a macrophage. Also provided are embodiments in which the one or more symptoms of the microbial infection comprise microbe-induced apoptosis.

In addition, the present invention provides methods of treating microbial infection of a cell, comprising, providing: i) a cell suspected of having a microbial infection, wherein the cell comprises an anti-apoptotic gene; and ii) a composition comprising an agent for increasing activity of the anti-apoptotic gene; and contacting the cell with the composition under conditions such that expression of the anti-apoptotic gene of the cell is increased. In some embodiments, the cell is in a population of cells, a tissue or an animal. In a subset of these embodiments, the animal is a human or other mammal. In particularly preferred embodiments, the microbial infection comprises a bacterial infection. In a subset of these embodiments, the bacterial infection comprises an infection with bacteria selected from the group consisting of Bacillus species, Escherichia species, Salmonella species, Shigella species, Yersinia species, Listeria species, Legionella species, Mycobacterium species, Streptococcus species and Haemophilus species. In some preferred embodiments, the agent comprises one or more of a 24(S),25-epoxycholesterol (EC), T1317, GW3965, and 9-cis-retinoic acid (9cRA). Moreover, in some preferred embodiments, the cell is a myeloid cell, such as a macrophage. Also provided are embodiments in which the anti-apoptotic gene comprises one or more AIM, Birc1a, and Bcl-X_{L}.

The present invention further provides methods for treating microbial infection of a cell, comprising: providing: i) a cell suspected of having a microbial infection, wherein the cell comprises a pro-apoptotic gene; and ii) a composition comprising an agent for decreasing activity of the pro-apoptotic gene; and contacting the cell with the composition under conditions such that expression of the pro-apoptotic gene of the cell is decreased. In some embodiments, the cell is in a population of cells, a tissue or an animal. In a subset of these embodiments, the animal is a human or other mammal. In particularly preferred embodiments, the microbial infection comprises a bacterial infection. In a subset of these embodiments, the bacterial infection comprises an infection with bacteria selected from the group consisting of Bacillus species, Escherichia species, Salmonella species, Shigella species, Yersinia species, Listeria species, Legionella species, Mycobacterium species, Streptococcus species and Haemophilus species. In some preferred embodiments, the agent comprises one or more of a 24(S),25-epoxycholesterol (EC), T1317, GW3965, and 9-cis-retinoic acid (9cRA). Moreover, in some preferred embodiments, the cell is a myeloid cell, such as a macrophage. Also provided are embodiments in which the pro-apoptotic gene comprises one or more deoxyribonuclease I-like 3 (Dnase1L3), Caspase 1, Caspase 4, Caspase 7, Caspase 11, Caspase 12, Fas ligand, cell death-inducing DFFA-like effector A (CIDE-A), and peptidoglycan recognition protein (Tag7).

### BRIEF DESCRIPTION OF THE DRAWINGS

FIG. 1 shows exemplary embodiments in which LXR agonists and RXR agonists inhibit apoptotic responses to growth factor withdrawal and protein synthesis inhibition.

FIG. 2 shows exemplary embodiments demonstrating that LXR and RXR activation protects macrophages from pathogen-induced apoptosis.

FIG. 3 shows an exemplary embodiment demonstrating time requirements for effects of LXR/RXR agonists on macrophage survival and identification of candidate genes.

FIG. 4 shows an exemplary embodiment in which activation of LXR antagonizes the pro-apoptotic program induced by engagement of TLR4.

FIG. 5 shows an exemplary embodiment in which Apoptosis Inhibitor expressed by Macrophages (AIM) is synergistically induced by LXR and RXR agonists, thereby contributing to their anti-apoptotic effects.

FIG. 6 provides the mouse LXR-alpha nucleic acid (SEQ ID NO:4) and amino acid (SEQ ID NO:5) sequences in panels A and B, respectively.

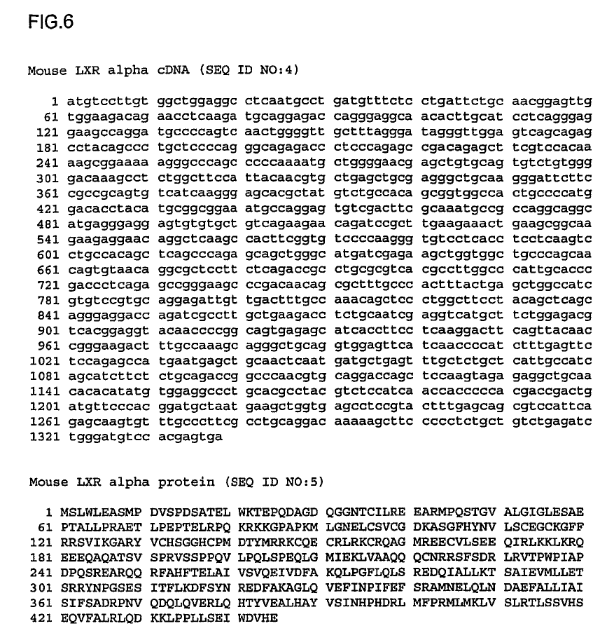

FIG. 7 provides the mouse LXR-beta nucleic acid (SEQ ID NO:6) and amino acid (SEQ ID NO:7) sequences in panels A and B, respectively.

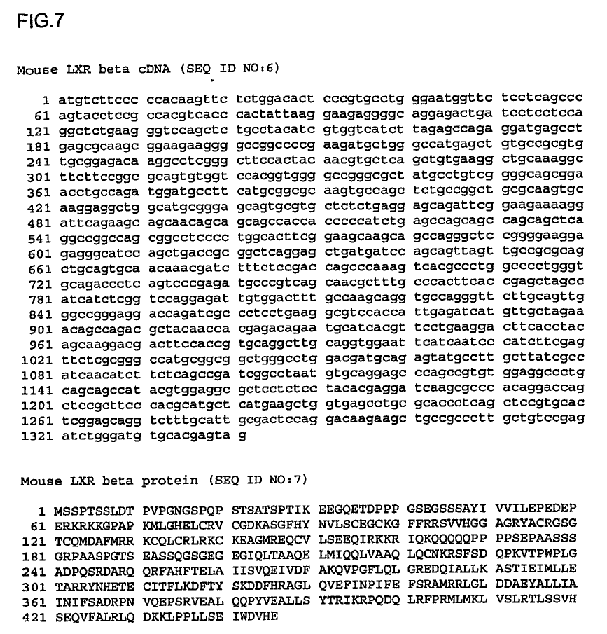

FIG. 8 provides the human LXR-alpha nucleic acid (SEQ ID NO:8) and amino acid (SEQ ID NO:9) sequences in panels A and B, respectively.

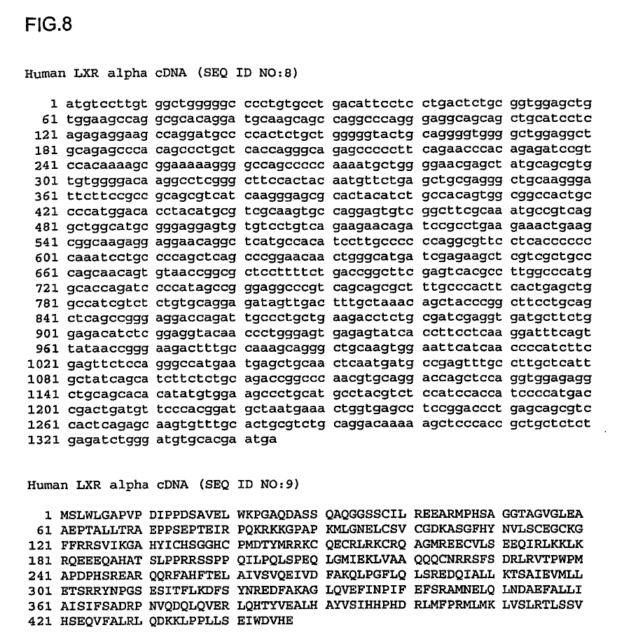

FIG. 9 provides the human LXR-beta nucleic acid (SEQ ID NO:10) and amino acid (SEQ ID NO: 11) sequences in panels A and B, respectively.

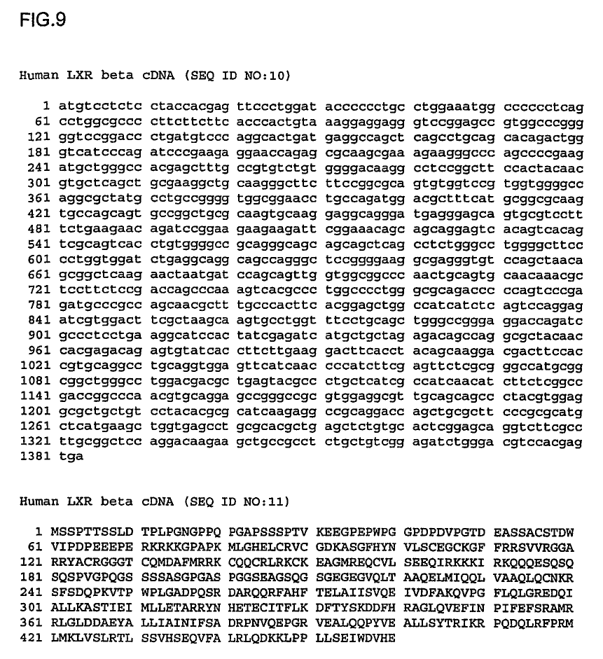

### DEFINITIONS

To facilitate understanding of the invention, a number of terms are defined.

As used herein including within this specification and the appended claims, the forms “a,” “an” and “the” includes both singular and plural references unless the content clearly dictates otherwise.

As used herein, the term “or” when used in the expression “A or B,” and where A and B refer to a composition, disease, product, etc., means one, or the other, or both.

As used herein, the terms “microorganism” and “microbe” refer to any organism of microscopic or ultramicroscopic size including, but not limited to, viruses, bacteria, fungi and protozoa.

Viruses are exemplified by, but not limited to, Arenaviridae, Baculoviridae, Birnaviridae, Bunyaviridae, Cardiovirus, Corticoviridae, Cystoviridae, Epstein-Barr virus, Filoviridae, Hepadniviridae, Hepatitis virus, Herpesviridae, Influenza virus, Inzoviridae, Iridoviridae, Metapneumovirus, Orthomyxoviridae, Papovavirus, Paramyxoviridae, Parvoviridae, Polydnaviridae, Poxyviridae, Reoviridae, Rhabdoviridae, Semliki Forest virus, Tetraviridae, Toroviridae, Vaccinia virus, Vesicular stoimatitis virus, togaviruses, flaviviruses, coronaviruses, and picornaviruses (including Adenovirus, Enterovirus, Immunodeficiency virus, Poliovirus, and Retrovirus).

The term “bacteria” and “bacterium” refer to all prokaryotic organisms, including those within all of the phyla in the Kingdom Procaryotae. It is intended that the term encompass all microorganisms considered to be bacteria including but not limited to, Mycoplasina species, Chlamydia species, Actinomyces species, Streptomyces species, Rickettsia species, Enterobacteriaceae species, Escherichia species and Enterococcus species. All forms of bacteria are included within this definition including cocci, bacilli, spirochetes, spheroplasts, protoplasts, etc. and are exemplified by Escherichia coli, Haemophilus influenza, Mycobacterium tuberculosis, Neisseria gonorrhoeae, Pseudomonas aeruginosa, Shigella dysenteriae, Staphylococcus aureus, and Streptococcus pneumonia. Also included within these terms are prokaryotic organisms that are gram negative or gram positive. “Gram-negative” and “gram-positive” refer to staining patterns with the Gram-staining process that is well known in the art (Finegold and Martin, Diagnostic Microbiology, 6th Ed. (1982), CV Mosby St. Louis, pp 13-15). “Gram-positive bacteria” are bacteria that retain the primary dye used in the Gram stain, causing the stained cells to appear dark blue to purple under the microscope. Exemplary gram-positive bacteria include Staphylococcus aureus, Staphylococcus hemolyticus, and Streptococcus pneumoniae. “Gram-negative bacteria” do not retain the primary dye used in the Gram stain, but are stained by the counterstain. Thus, gram-negative bacteria appear red. Exemplary gram-negative bacteria include Escherichia coli, Klebsiella pneumoniae, Pseudomonas aeruginosa, Haemophilus influenzae, and Neisseriae gonorrhoeae.

As used herein, the term “pathogen” refers to any microbe that is associated with infection, inflammation and disease. It is not meant to limit the pathogen to those traditionally considered bacterial pathogens (e.g., B. anthracis, Y. pseudotuberculosis, S. typhimurium, K. pneumoniae, H. Influenza, S. aureus, S. pyogenes, S. dysenteriae, S. flexneri, etc.) or opportunistic bacterial pathogens (e.g., P. aeruginosa, S. marcesens, S. mitis, etc.) or a viral pathogen (Influenzavirus).

The terms “fungi” and “yeast” are used interchangeably herein and refer to the art recognized group of eukaryotic protists known as fungi. “Yeast” as used herein can encompass the two basic morphologic forms of yeast and mold and dimorphisms thereof. Exemplary fungal species include Aspergillus species (such as Aspergillus fumigatus, Aspergillus flavus, Aspergillus niger, Aspergillus nidulans, and Aspergillus terreus), Blastomyces species, Candida species (such as Candida albicans, Candida stellatoidea, Candida glabrata, Candida tropicalis, Candida parapsilosis, Candida krusei, Candida pseudotropicalis, Candida guilliermondii, and Candida rugosa), Coccidioides species, Cryptococcus species, Epidermophyton species, Hendersonula species, Histoplasma species, Microsporum species, Paecilomyces species, Paracoccidioides species, Pneuinocystis species such as Pneumocystis carinii, Trichophyton species, and Trichosporium species. Exemplary fungi include Pneumocystis carinii, Cryptococcus neoformans, Histoplasma capsulatuin, Coccidioides immitis, and Pneumocystis carinii.

The term “protozoa” refers to the phylum of animals that have an essentially acellular structure through varying from simple uninucleate protoplasts (as most amoebas) to cell colonies (such as volvox), syncytia (such as pelomyxa), or highly organized protoplasts (such as various higher ciliates) that are more complex in organization and differentiation than most metazoan cells. Exemplary parasitic protozoa include the Plasmodium species (such as Plasmodium vivax, Plasmodium falciparum, Plasmodium ovale and Plasmodium malariae), Leishmania species, Toxoplasma gondii, Trypanosoma cruzi, Pneumocystis carinii, Entameba histolytica, Cryptosporidium parvui, Giardia lamblia, and amoebae. Parasitic protozoa also infect non-human animals such as fish. Protozoans can infect both external and internal portions of the fish including the gills, fins, skin, and digestive organs. External protozoa of major concern to aquaculturists include members of the genus Costia, Chilodon, Scyphidia, Trichodina, Epistylis, Carchesium, and Trichophrya. The external ciliate, Ichthyophthirius multifiliis, causes white spot disease known as Ick, which is difficult to control and is often observed in crowded cultures of catfish and warm-water aquarium fish.

As used herein, the terms “infecting” and “infection” with a microorganism (such as a bacterium or virus) refer to co-incubation of a biological sample, (e.g., cell, tissue, etc.) with a microorganism under conditions such that the microorganism enters, invades, or inhabits one or more cells of the biological sample. In some embodiments, the term infection refers to co-incubation of a biological sample with a microorganism under conditions such that nucleic acid sequences contained within the microorganism are introduced into one or more cells of the biological sample. In some embodiments, all or essentially all of the microorganism is introduced into the one or more cells. Infection may be in vitro and/or in vivo.

As used herein, the terms “administering” and “administered,” refer to giving to and/or applying, e.g. meting out, dispensing, such as giving to a cell or a patient and/or applying, e.g., as a remedy, (for example, administering a sedative, or administering first aid). In some embodiments, the composition(s) of the present invention is/are administered to one or more of the cell, tissue, patient, in a single dose, while in other embodiments, the composition is administered to one or more of the cell, tissue, patient, in multiple doses. In some embodiments, the administering is selected from the group consisting of administration in a fluid, in cell medium, in a growth chamber, in an assay plate, in a test tube, and the like. In some embodiments, the administering is selected from the group consisting of subcutaneous injection, oral administration, intravenous administration, intraarterial administration, intraperitoneal administration, rectal administration, vaginal administration, topical administration, intramuscular administration, intranasal administration, intrapulmonary administration (e.g., inhalation, insufflation, etc.), intratracheal administration, epidermal administration, transdermal administration, subconjunctival administration, intraocular administration, periocular administration, retrobulbar administration, subretinal administration, suprachoroidal administration, intramedullar administration, intracranial administration, intraventricular administration, and intrathecal administration. In alternative embodiments, the administering is administration from a source selected from the group consisting of mechanical reservoirs, devices, implants, and patches. In still further embodiments, the composition is in a form selected from the group consisting of pills, capsules, liquids, gels, powders, suppositories, suspensions, creams, jellies, aerosol sprays, and dietary supplements. Additionally, peptide(s) and peptidomimetic(s) may be administered as an ointment, lotion or gel (i.e., for the treatment of skin and mucosal areas). In some embodiments, it is expected that cells in a tissue will contain an expression vector and express a gene of interest (i.e., such that the peptide(s) and peptidomimetic(s) of interest are expressed in the tissue(s)).

As used herein, the term “contacting” cells with an agent or microbe refers to placing the agent or a microbe in a location that will allow it to touch the cell in order to produce “contacted” cells. The contacting may be accomplished using any suitable method. For example, in one embodiment, contacting is by adding the agent or a microbe to a tube of cells. Contacting may also be accomplished by adding the agent to a culture of the cells. In another embodiment, contacting may be accomplished by administration of the agent or microbe to an animal in vivo.

As used herein, the terms “anti-bacterial” and “antimicrobial” refer to any agent that reduces the growth of (including killing) microbes. It is intended that the term be used in its broadest sense, and includes, but is not limited to, agents described herein, for example those which are produced naturally or synthetically.

As used herein, the terms “antigen,” “immunogen,” “antigenic,” “immunogenic,” “antigenically active,” and “immunologically active” refer to any substance that is capable of inducing a specific humoral or cell-mediated immune response. An immunogen generally contains at least one epitope. Immunogens are exemplified by, but not restricted to molecules, which contain a peptide, polysaccharide, nucleic acid sequence, and/or lipid. Complexes of peptides with lipids, polysaccharides, or with nucleic acid sequences are also contemplated, including (without limitation) glycopeptide, lipopeptide, glycolipid, etc. These complexes are particularly useful immunogens where smaller molecules with few epitopes do not stimulate a satisfactory immune response by themselves.

As used herein, the terms “antigen-presenting cell” and “APC” refer to a term most commonly used when referring to white blood cells that present processed antigenic peptide and MHC class I and/or II molecules to the T-cell receptor on lymphocytes, (e.g. macrophages, dendritic cells, B-cells and the like). However, other non-white blood cells can also be referred to as “antigen-presenting cells” and more specifically “nonprofessional antigen presenting cell” since they present peptides within MHC class I and class II to T-cells and the like, e.g. as occurs with viral infected cells, cancer cells and the like.

As used herein, the terms “dendritic cell,” “DC,” and “professional antigen-presenting cells” can evoke an antigen response at least 10× greater in magnitude when compared to other APCs under similar conditions (reviewed in Mellman et al. (1998) Trends Cell Biol. 8:231-7).

As used herein, the term “cell” refers to a single cell as well as to a population of (i.e., more than one) cells. The population may be a pure population comprising one cell type. Alternatively, the population may comprise more than one cell type. In the present invention, there is no limit on the number of cell types that a cell population may comprise.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “mixed cell culture,” refers to a mixture of two or more types of cells. In some embodiments, the cells are cell lines that are not genetically engineered, while in other embodiments the cells are genetically engineered cell lines. In some embodiments, the cells contain genetically engineered molecules. The present invention encompasses any combination of cell types suitable for the detection, identification, and/or quantitation of apoptosis in samples, including mixed cell cultures in which all of the cell types used are not genetically engineered, mixtures in which one or more of the cell types are genetically engineered and the remaining cell types are not genetically engineered, and mixtures in which all of the cell types are genetically engineered.

As used herein, the term “primary cell” is a cell that is directly obtained from a tissue (e.g. blood) or organ of an animal in the absence of culture. Typically, though not necessarily, a primary cell is capable of undergoing ten or fewer passages in vitro before senescence and/or cessation of proliferation. In contrast, a “cultured cell” is a cell that has been maintained and/or propagated in vitro for ten or more passages.

As used herein, the term “cultured cells” refer to cells that are capable of a greater number of passages in vitro before cessation of proliferation and/or senescence when compared to primary cells from the same source. Cultured cells include “cell lines” and “primary cultured cells.”

As used herein, the term “cell line,” refers to cells that are cultured in vitro, including primary cell lines, finite cell lines, continuous cell lines, and transformed cell lines, but does not require, that the cells be capable of an infinite number of passages in culture. Cell lines may be generated spontaneously or by transformation.

As used herein, the terms “primary cell culture,” and “primary culture,” refer to cell cultures that have been directly obtained from cells in vivo, such as from animal or insect tissue. These cultures may be derived from adults as well as fetal tissue.

As used herein, the terms “monolayer,” “monolayer culture,” and “monolayer cell culture,” refer to a cell that has adhered to a substrate and grow as a layer that is one cell in thickness. Monolayers may be grown in any format, including but not limited to flasks, tubes, coverslips (e.g., shell vials), roller bottles, etc. Cells may also be grown attached to microcarriers, including but not limited to beads.

As used herein, the term “suspension” and “suspension culture” refers to cells that survive and proliferate without being attached to a substrate. Suspension cultures are typically produced using hematopoietic cells, transformed cell lines, and cells from malignant tumors.

As used herein, the terms “culture media,” and “cell culture media,” refers to media that are suitable to support the growth of cells in vitro (i.e., cell cultures). It is not intended that the term be limited to any particular culture medium. For example, it is intended that the definition encompass outgrowth as well as maintenance media. Indeed, it is intended that the term encompass any culture medium suitable for the growth of the cell cultures of interest.

As used herein the term, the term “in vitro” refers to an artificial environment and to processes or reactions that occur within an artificial environment. In vitro environments exemplified, but are not limited to, test tubes and cell cultures. The term “in vivo” refers to the natural environment (e.g., an animal or a cell) and to processes or reactions that occur within a natural environment.

As used herein, the term “proliferation” refers to an increase in cell number.

As used herein, the term “differentiation” refers to the maturation process cells undergo whereby they develop distinctive characteristics, and/or perform specific functions, and/or are less likely to divide.

As used herein, the terms “isolated,” “to isolate,” “isolation,” “purified,” “to purify,” “purification,” and grammatical equivalents thereof as used herein, refer to the reduction in amount of at least one contaminant (such as protein and/or nucleic acid sequence) from a sample. Thus, purification results in “enrichment,” i.e., an increase in the amount of a desirable protein and/or nucleic acid sequence in the sample.

As used herein, the term “amino acid sequence” refers to an amino acid sequence of a naturally occurring or engineered protein molecule. “Amino acid sequence” and like terms, such as “polypeptide,” “peptide” or “protein” are not meant to limit the amino acid sequence to the complete, native amino acid sequence associated with the recited protein molecule.

As used herein, the terms “Liver X Receptor” and “LXR” refer to membrane spanning proteins that are members of the nuclear receptor superfamily, regulated by oxidized forms of cholesterol (oxysterols) and intermediate products of the cholesterol biosynthetic pathway (Janowski et al. (1996) Nature 383, 728-731; and Janowski et al. (1999) Proc Natl Acad Sci USA 96, 266-71). Two LXR isoforms, LXRα (NR1H3) and β (NR1H2), are encoded by distinct genes. In one embodiment, LXR is a monomer. It is not intended that LXR activity is limited to one LXR molecule. In one embodiment, LXR is an alternatively spliced molecule. In one embodiment, LXR is a heterodimer with RXR. In one embodiment, LXR is autophosphorylated.

As used herein, the terms “LXR-alpha” and “liver X receptor-alpha” refer to a human LXR-alpha gene and its gene product (e.g., Homo sapiens—GENBANK Accession No. NP_{—}005684), as well as its mammalian counterparts (including wild type and mutant products). Mammalian counterparts of human LXR-alpha include but are not limited to: Pan troglodytes (chimpanzee) GENBANK accession No. XP_{—}521906; Mus musculus (mouse) GENBANK Accession No. NP_{—}038867; Rattus norvegicus (rat) GENBANK Accession No. NP_{—}113815; Canis familiaris (dog) GENBANK accession No. XP_{—}540745; and Gallus gallus (chicken) GENBANK accession No. NP_{—}989873.

As used herein, the terms “LXR-beta” and “liver X receptor-beta” refer to a human LXR-beta gene and its gene product (e.g., Homo sapiens—GENBANK Accession No. NP_{—}009052), as well as its mammalian counterparts (including wild type and mutant products). Mammalian counterparts of human LXR-beta include but are not limited to: Mus musculus (mouse) GENBANK Accession No. NP_{—}033499; Rattus norvegicus (rat) GENBANK Accession No. NP_{—}113814; Canis familiaris (dog) GENBANK accession No. XP_{—}851316.

As used herein, the terms “retinoid X receptor” and “RXR” refer to members of the nuclear receptor superfamily that can be regulated by 9-cis retinoic acid (9cRA) and long chain polyunsaturated fatty acids (Heyman et al. (1992) Cell 68, 397-406; Chambon (1996) FASEB J 10, 940-954; Bourguet et al. (2000) Molecular Cell 5, 289-298; and Mata de Urquiza et al. (2000) Science 290, 2140-4). In one embodiment, RXR is a monomer. It is not intended that RXR activity is limited to one RXR molecule. In one embodiment, RXR is an alternatively spliced molecule. In one embodiment, RXR is a heterodimer with LXR. In one embodiment, RXR is autophosphorylated. The terms “RXR” and “retinoid X receptor” refer to a human RXR gene and its gene product, as well as its mammalian counterparts (including wild type and mutant products). Mammalian counterparts of human RXR include but are not limited to nonhuman primate, rodent, dog, and chicken RXRs. The terms encompasses RXR α1, α2, β1, β2, γ1 and γ².

As used herein, the term “ligand” refers to a molecule that binds to a second molecule. A particular molecule may be referred to as either, or both, a ligand and second molecule. Examples of second molecules include a receptor of the ligand, and an antibody that binds to the ligand.

The terms “LXR agonist” and “liver X receptor agonist” as used herein, refer to any molecule that increases the expression of or activity of LXR. LXR agonists suitable for use in the methods and compositions of the present invention include but are not limited to 24(S),25-epoxycholesterol (EC), T1317, GW3965, GSK3987, 22-(R)-hydroxycholesterol, and T0901317.

The terms “RXR agonist” and “retinoid X receptor agonist” as used herein, refer to any molecule that increases the expression of or activity of RXR. An exemplary RXR agonist suitable for use in the methods and compositions of the present invention is 9-cis retinoic acid (9cRA).

In some embodiments, the agonist is a small molecule, a protein, a peptide, a peptidomimetic, or a nucleic acid. The term “small molecule” refers to a molecule having a molecular weight of less than 1,000 daltons. The terms “polypeptide” and “protein” are used interchangeably herein to refer to a polymer of 10 to more than 100 amino acid residues. The term “peptide” refers to a polymer of two to nine amino acids where the alpha carboxyl group of one is bound to the alpha amino group of another. The terms “peptide,” “polypeptide”, and “protein” apply to amino acid polymers in which one or more amino acid residue is an artificial chemical analogue of a corresponding naturally occurring amino acid, as well as to naturally occurring amino acid polymers. The term “peptidomimetic” refers to a compound containing non-peptidic structural elements that is capable of mimicking or antagonizing the biological action(s) of a natural peptide. The term “nucleic acid” refers to a linear polymer of nucleotides linked by 3′, 5′ phosphodiester linkages. In DNA (deoxyribonucleic acid), the sugar group is deoxyribose and the bases of the nucleotides are adenine, guanine, thymine and cytosine. In RNA (ribonucleic acid), the sugar group is ribose and uracil replaces thymine.

As is known in the art, “protein phosphorylation” is a common regulatory mechanism used by cells to selectively modify proteins carrying regulatory signals from outside the cell to the cytoplasm and ultimately the nucleus. The proteins that execute these biochemical modifications are a group of enzymes known as protein kinases. They may further be defined by the substrate residue that they target for phosphorylation. One group of protein kinases is the tyrosine kinases (TKs), which selectively phosphorylate a target protein on its tyrosine residues. Some tyrosine kinases are membrane-bound receptors (RTKs), and, upon activation by a ligand, can autophosphorylate as well as modify substrates. The initiation of sequential phosphorylation by ligand stimulation is a paradigm that underlies the action of such effectors as, for example, LPS, LTA, Lethal Toxin (LT), and interferons such as Interferon-β (IFN-β). The receptors for these ligands are tyrosine kinases and provide the interface between the binding of a ligand (hormone, growth factor) to a target cell and the transmission of a signal into the cell by the activation of one or more biochemical pathways. Ligand binding to a receptor tyrosine kinase activates its intrinsic enzymatic activity (See, e.g., Ullrich and Schlessinger (1990) Cell 61:203-212). Tyrosine kinases can also be cytoplasmic, non-receptor-type enzymes and act as a downstream component of a signal transduction pathway.

As used herein, the term “protein kinase” refers to a protein that catalyzes the addition of a phosphate group from a nucleoside triphosphate to an amino acid in a protein. Kinases comprise the largest known enzyme superfamily and vary widely in their target proteins. Kinases can be categorized as protein tyrosine kinases (PTKs), which phosphorylate tyrosine residues, and protein serine/threonine kinases (STKs), which phosphorylate serine and/or threonine residues and the like. Some kinases have dual specificity for both serine/threonine and tyrosine residues. Almost all kinases contain a conserved 250-300 amino acid catalytic domain. This domain can be further divided into 11 subdomains. N-terminal subdomains I-IV fold into a two-lobed structure that binds and orients the ATP donor molecule, and subdomain V spans the two lobes. C-terminal subdomains VI-XI bind the protein substrate and transfer the gamma phosphate from ATP to the hydroxyl group of a serine, threonine, or tyrosine residue. Each of the 11 subdomains contains specific catalytic residues or amino acid motifs characteristic of that subdomain. For example, subdomain I contains an 8-amino acid glycine-rich ATP binding consensus motif, subdomain II contains a critical lysine residue that contributes to maximal catalytic activity, and subdomains VI through IX comprise the highly conserved catalytic core. STKs and PTKs also contain distinct sequence motifs in subdomains VI and VIII, which may confer hydroxyamino acid specificity. Some STKs and PTKs possess structural characteristics of both families. In addition, kinases may also be classified by additional amino acid sequences, generally between 5 and 100 residues, which either flank or occur within the kinase domain.

Non-transmembrane PTKs form signaling complexes with the cytosolic domains of plasma membrane receptors. Receptors that signal through non-transmembrane PTKs include cytokine, hormone, and antigen-specific lymphocytic receptors. Many PTKs were first identified as oncogene products in cancer cells in which PTK activation was no longer subject to normal cellular controls. In fact, about one third of the known oncogenes encode PTKs. Furthermore, cellular transformation (oncogenesis) is often accompanied by increased tyrosine phosphorylation activity (See, e.g., Carbonneau and Tonks (1992) Annu Rev Cell Biol 8:463-93). Regulation of PTK activity may therefore be an important strategy in controlling some types of cancer.

Examples of protein kinases include, but are not limited to, cAMP-dependent protein kinase, protein kinase C, and cyclin-dependent protein kinases (See, e.g., U.S. Pat. Nos. 6,034,228; 6,030,822; 6,030,788; 6,020,306; 6,013,455; 6,013,464; and 6,015,807, all of which are incorporated herein by reference).

As used herein, the term “protein phosphatase” refers to proteins that remove a phosphate group from a protein. Protein phosphatases are generally divided into two groups, receptor-type and non-receptor type (e.g. intracellular) proteins. An additional group includes dual specificity phosphatases. Most receptor-type protein tyrosine phosphatases contain two conserved catalytic domains, each of which encompasses a segment of 240 amino acid residues (See e.g., Saito et al. (1991) Cell Growth and Diff 2:59). Receptor protein tyrosine phosphatases can be subclassified further based upon the amino acid sequence diversity of their extracellular domains (See e.g., Krueger et al. (1992) Proc Natl Acad Sci USA 89:7417-7421). Examples of protein phosphatases include, but are not limited to, human protein phosphatase (PROPHO), FIN13, cdc25 tyrosine phosphatase, protein tyrosine phosphatase (PTP) 20, PTP 1D, PTP-D1, PTP .t., PTP-S31 (See e.g., U.S. Pat. Nos. 5,853,997; 5,976,853; 5,294,538; 6,004,791; 5,589,375; 5,955,592; 5,958,719; and 5,952,212; all of which are incorporated herein by reference).

As used herein, the term “activating” when in reference to a biochemical response

(such as kinase activity) and/or cellular response (such as cell proliferation) refers to increasing the biochemical and/or cellular response.

As used herein, the term “activated” when in reference to a cell, refers to a cell that has undergone a response that alters its physiology and shifts it towards making a biologically response and becoming biologically “active” hence “activated.” For example, a monocyte becomes activated to mature into a macrophage. For another example, a macrophage becomes activated upon contact with an endotoxin (such as LPS) wherein the activated macrophage can produce an increased level and/or type of a molecule associated with activation (e.g. iNOS, MMP-12 Metalloelastase and the like). In another example, an immature dendritic cell becomes activated to mature into a functional dendritic cell. An “activated” cell does not necessarily, although it may, undergo growth or proliferation. Typically, activation of macrophages and DCs, unlike lymphocytes such as T-cells, B-cells and the like, does not stimulate proliferation. Activation can also induce cell death such as in activation-induced cell death (AICD) of T cells. In one embodiment of the present invention, activation can lead to apoptotic death.

As used herein, the terms “naturally occurring,” “wild-type” and “wt” as used herein when applied to a molecule or composition (such as nucleotide sequence, amino acid sequence, cell, apoptotic blebs, external phosphatidylserine, etc.), mean that the molecule or composition can be found in nature and has not been intentionally modified by man. For example, a naturally occurring polypeptide sequence refers to a polypeptide sequence that is present in an organism that can be isolated from a source in nature, wherein the polypeptide sequence has not been intentionally modified by man.

The terms “derived from” and “established from” when made in reference to any cell disclosed herein refer to a cell which has been obtained (e.g., isolated, purified, etc.) from the parent cell in issue using any manipulation, such as, without limitation, infection with virus, transfection with DNA sequences, treatment and/or mutagenesis using for example chemicals, radiation, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the term “biologically active,” refers to a molecule (e.g. peptide, nucleic acid sequence, carbohydrate molecule, organic or inorganic molecule, and the like) having structured, regulatory, aid/or biochemical functions.

As used herein, the term “apoptosis” refers to the process of non-necrotic cell death that takes place in metazoan animal cells following activation of an intrinsic cell suicide program. Apoptosis is a normal process in the proper development and homeostasis of metazoan animals and usually leads to cell death. Apoptosis is also triggered pathologically by microbial infections resulting in increasing susceptibility to apoptosis and/or outright death. Apoptosis involves sequential characteristic morphological and biochemical changes. One early marker of apoptosis is the flipping of plasma membrane phosphatidylserine, inside to outside, with cellular blebbing called “zeiosis,” of plasma membrane releasing vesicles containing cellular material including RNA and DNA as apoptotic bodies. During apoptosis, there is cell expansion followed by shrinkage through release of apoptotic bodies and lysis of the cell, nuclear collapse and fragmentation of the nuclear chromatin, at certain intranucleosomal sites, due to activation of endogenous nucleases. Apoptotic bodies are typically phagocytosed by other cells, in particular immunocytes such as monocytes, macrophages, immature dendritic cells and the like. One of skill in the art appreciates that reducing the ability to undergo apoptosis results in increased cell survival, without necessarily (although it may include) increasing cell proliferation. Accordingly, as used herein, the terms “reduce apoptosis” and “increase survival” are equivalent. In addition, as used herein, the tenns “increase apoptosis” and “reduced survival” are equivalent.

Apoptosis may be determined but not limited to the assays described herein and include methods known in the art. For example, apoptosis may be determined by techniques for detecting DNA fragmentation, (for example any version of the Terminal deoxynucleotidyl transferase (TdT)-mediated dUTP Nick End-Labeling TUNEL technique (Gavrieli et al. (1992) J Cell Biol. 119:493-501), nuclear staining with nucleic acid dyes such as Hoechst 33342, Acridine Orange and the like, and detecting DNA “ladder” fragmentation patterns associated with apoptosis (e.g. DNA gels and the like)). In one embodiment, apoptosis is measured by TUNEL, while in another embodiment, apoptosis is measured by observing DNA fragmentation in a ladder pattern (for example, Park et al. (2002) Science 297, 2048-51). Apoptosis may be determined by morphological measurements including but not limited to measuring live cells, early apoptotic cells, late apoptotic cells and cell death via apoptosis. For example, the cells' increased display of externally flipped phosphatidylserine, an early indicator of apoptosis, binds external Annexin-V. Thus Annexin-V attached to fluorescent molecules can be used to stain non permeabilized cells and often further combined with vital dyes (example propidium Iodide (PI), Etbidium Bromide (EtBr) and the like) allowing fluorescent activated cell sorting (FACS) analysis measuring of live, early apoptotic, late apoptotic and dead cells (Ozawa et al. (1999) J Exp Med 189:711-8). Further, general live versus dead cell assays may also be employed, for example double staining with EtBr and Calcein AM for live microscopy determinations and FACS. Apoptosis may be determined by the presence of molecular fragments in apoptotic cells not present in live non-apoptotic cells. For example, caspase molecules such as Caspases-3,6,7, and 9 and the like, are cleaved during apoptotic processes, release of cytochrome c, PARP (poly(ADP-ribose) polymerase) cleavage, and the like. Thus detecting the increased presence of predictable sizes of cleaved caspase subunits in apoptotic cells as compared to non-apoptotic cells indicate that cells are apoptotic. Furthermore, apoptosis may be monitored by changes in protein activity of molecules that decrease or increase cell survival and/or proliferation. For example, protein kinases and nuclear factors increase in activity during apoptosis and serve to either contribute to the apoptotic process or protect against apoptotic damage.

As used herein, the term “cellular response” refers to an increase or decrease of activity by a cell. For example, the “cellular response” may constitute but is not limited to apoptosis, death, DNA fragmentation, blebbing, proliferation, differentiation, adhesion, migration, DNA/RNA synthesis, gene transcription and translation, and/or cytokine secretion or cessation of such processes. A “cellular response” may comprise an increase or decrease of dephosphorylation, phosphorylation, calcium flux, target molecule cleavage, protein-protein interaction, nucleic acid-nucleic acid interaction, and/or protein/nucleic acid interaction and the like. As used herein, the term “target molecule cleavage” refers to the splitting of a molecule (for example in the process of apoptosis, cleavage of pro-caspases into fragments, cleavage of DNA into predicable sized fragments and the like). As used herein, the term “interaction” refers to the reciprocal action or influence of two or more molecules on each other.

As used herein, the term “transgenic” when used in reference to a cell refers to a cell which contains a transgene, or whose genome has been altered by the introduction of a transgene. The term “transgenic” when used in reference to a tissue refers to a tissue, which comprises one or more cells that contain a transgene, or whose genome has been altered by the introduction of a transgene. Transgenic cells, and tissues may be produced by several methods including the introduction of a “transgene” comprising nucleic acid (usually DNA) into a target cell or integration of the transgene into a chromosome of a target cell by way of human intervention, such as by the methods described herein.

As used herein, the term “transgene” as used herein refers to any nucleic acid sequence that is introduced into the cell by experimental manipulations. A transgene may be an “endogenous DNA sequence” or a “heterologous DNA sequence” (i.e., “foreign DNA”). The term “endogenous DNA sequence” refers to a nucleotide sequence that is naturally found in the cell into which it is introduced so long as it does not contain some modification (e.g., a point mutation, the presence of a selectable marker gene, etc.) relative to the naturally-occurring sequence. Examples of Toll-like receptor 4 mutations and variants, herein incorporated by reference, are shown in U.S. Pat. No. 6,740,487, U.S. Patent Appln. No., 20020173001A1; mutations associated with atherosclerosis in U.S. Patent Appln. No., 20030232352A1, PCT publication WO03/050137 and PCT publication WO03/035110. The term “heterologous DNA sequence” refers to a nucleotide sequence that is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes an endogenous DNA sequence that contains some modification. Generally, although not necessarily, heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

As used herein, the terms “agent,” “test agent,” “molecule,” “test molecule,” “compound,” and “test compound” as used interchangeably herein, refer to any type of molecule (for example, a peptide, nucleic acid, carbohydrate, lipid, organic molecule, and inorganic molecule, etc.) any combination molecule for example glycolipid, etc.) obtained from any source (for example, plant, animal, protist, and environmental source, etc.), or prepared by any method (for example, purification of naturally occurring molecules, chemical synthesis, and genetic engineering methods, etc.). Test agents are exemplified by, but not limited to individual and combinations of antibodies, chimeric molecules (for example, herein incorporated by reference, U.S. Patent Appln. No., 20040009167A1), nucleic acid sequences, and other agents as further described below.

In one embodiment, the term “test agent” refers to any chemical entity, pharmaceutical, drug, and the like that can be used to treat or prevent a disease, illness, sickness, or disorder of bodily function. Test agents comprise both known and potential therapeutic agents. A test agent can be determined to be therapeutic by screening using the screening methods of the present invention. A “known therapeutic agent” refers to a therapeutic agent that has been shown (e.g., through animal trials or prior experience with administration to humans) to be effective in such treatment or prevention. In other words, a known therapeutic agent is not limited to an agent efficacious in the treatment of disease (e.g., cancer). Agents are exemplified by, but not limited to, antibodies, nucleic acid sequences such as ribozyme sequences, and other agents as further described herein. Examples of using Retinoid X Receptor inhibitors, herein incorporated by reference, are shown in U.S. Patent Appln. Nos., 20030077279A1; 20020192217A1. Examples of identifying agents for an anti-tumor PKR assay are described in U.S. Pat. No. 5,670,330.

The test agents identified by and/or used in the invention's methods include any type of molecule (for example, a peptide, nucleic acid, carbohydrate, lipid, organic, and inorganic molecule, etc.) obtained from any source (for example, plant, animal, and environmental source, etc.), or prepared by any method (for example, purification of naturally occurring molecules, chemical synthesis, and genetic engineering methods, etc.).

The terms “chosen from A, B and C” and “chosen from one or more of A, B and C” are equivalent terms that mean selecting any one of A, B, and C, or any combination of A, B, and C.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b, etc.

Unless otherwise indicated, all numbers expressing quantities of ingredients, properties such as molecular weight, reaction conditions, and so forth as used herein, are to be understood as being modified in all instances by the term “about.” Accordingly, unless indicated to the contrary, the numerical parameters herein are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

The term “not” when preceding, and made in reference to, any particularly named molecule (e.g., nucleic acid sequence, protein sequence, apoptotic blebs, external phosphatidylserine, etc.), and/or phenomenon (e.g., apoptosis, cell death, cell survival, cell proliferation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) means that only the particularly named molecule or phenomenon is excluded.

The terms “altering” and “modulating” and grammatical equivalents as used herein in reference to the level of any molecule (e.g., nucleic acid sequence, protein sequence, apoptotic blebs, external phosphatidylserine, etc.), and/or phenomenon (e.g., apoptosis, cell death, cell survival, cell proliferation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) refer to an increase and/or decrease (measurable change) in the quantity of the molecule and/or phenomenon, regardless of whether the quantity is determined objectively, and/or subjectively. In some preferred embodiments, the quantity of molecule and/or phenomenon in the first sample is at least 10%, 25%, 50%, 75%, 90%, or 95% different than the quantity of the same molecule and/or phenomenon in a second sample.

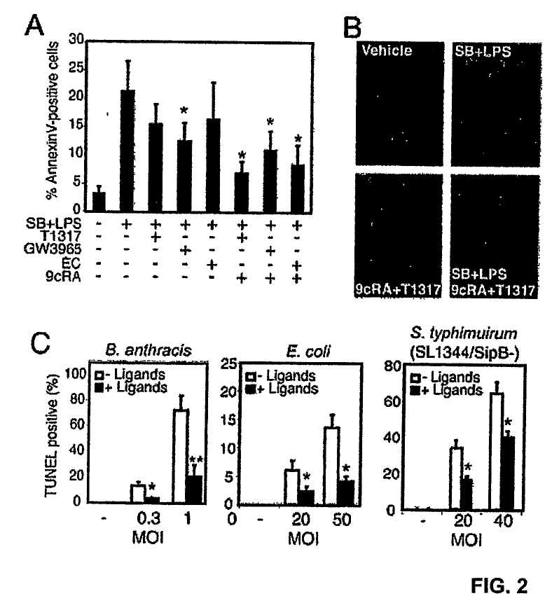

Unless defined otherwise in reference to the level of molecules and/or phenomena, the terms “reduce,” “inhibit,” “diminish,” “suppress,” “decrease,” and grammatical equivalents (for example, reducing, reduced, and the like) when in reference to the level of any molecule (e.g., nucleic acid sequence, protein sequence, apoptotic blebs, external phosphatidylserine, etc.), and/or phenomenon (e.g., apoptosis, cell death, cell survival, cell proliferation, caspase cleavage, receptor dimerization, receptor complex formation, phosphorylation, DNA fragmentation, molecule translocation, binding to a molecule, expression of a nucleic acid sequence, transcription of a nucleic acid sequence, enzyme activity, etc.) in a first sample relative to a second sample, mean that the quantity of molecule and/or phenomenon in the first sample is lower than in the second sample by a measurable amount (or by an amount that is statistically significant using any art-accepted statistical method of analysis). In one embodiment, the reduction may be determined subjectively, for example, when a patient refers to their subjective perception of disease symptoms, such as pain, difficulty in breathing, clarity of vision, nausea, tiredness, etc. In some preferred embodiments, the quantity of molecule and/or phenomenon in the first sample is at least 10%, 25%, 50%, 75%, 90%, or 95% lower than the quantity of the same molecule and/or phenomenon in a second sample. In one embodiment, the reduction may be determined subjectively, for example when comparing DNA fragmentation (e.g. FIG. 2b and the like) etc.

Unless defined otherwise in reference to the level of molecules and/or phenomena, the terms “increase,” “elevate,” “raise,” and grammatical equivalents when in reference to the level of any molecule (e.g., nucleic acid sequence, protein sequence, apoptotic blebs, external phosphatidylserine, etc.), and/or phenomenon (e.g., apoptosis, cell death, cell survival, cell proliferation, caspase cleavage, receptor dimerization, receptor complex formation, DNA fragmentation, molecule translocation, binding to a molecule, expression of a nucleic acid sequence, transcription of a nucleic acid sequence, enzyme activity, etc.) in a first sample relative to a second sample, mean that the quantity of the molecule and/or phenomenon in the first sample is higher than in the second sample by any amount that is statistically significant using any art-accepted statistical method of analysis. In one embodiment, the increase may be determined subjectively, for example when a patient refers to their subjective perception of disease symptoms, such as pain, difficulty in breathing, clarity of vision, nausea, tiredness, etc. In some preferred embodiments, the quantity of molecule and/or phenomenon in the first sample is at least 10%, 25%, 50%, 75%, 90%, or 95% higher than the quantity of the same molecule and/or phenomenon in a second sample.

Reference herein to any specifically named protein (such as Liver X Receptor, Retinoid X Receptor, etc.) refers to any and all equivalent fragments, fusion proteins, and variants of the specifically named protein, having at least one of the biological activities (such as those disclosed herein and/or known in the art) of the specifically named protein, wherein the biological activity is detectable by any method.

The term “fragment” when in reference to a protein (such as Liver X Receptor, Retinoid X Receptor, etc.) refers to a portion of that protein that may range in size from four (4) contiguous amino acid residues to the entire amino acid sequence minus one amino acid residue. Thus, a polypeptide sequence comprising “at least a portion of an amino acid sequence” comprises from four (4) contiguous amino acid residues of the amino acid sequence to the entire amino acid sequence.

The term “fusion protein” refers to two or more polypeptides that are operably linked. The term “operably linked” when in reference to the relationship between nucleic acid sequences and/or amino acid sequences refers to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest. The term also refers to the linkage of amino acid sequences in such a manner so that a functional protein is produced.

The term “variant” of a protein (such as Liver X Receptor, Retinoid X Receptor, etc.) as used herein is defined as an amino acid sequence, which differs by insertion, deletion, and/or conservative substitution of one or more amino acids from the protein of which it is a variant. The term “conservative substitution” of an amino acid refers to the replacement of that amino acid with another amino acid, which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains, which may be conservatively substituted one for the other include phenylalanine, tyrosine, and tryptophan. Cysteine and methionine are sulphur-containing amino acids, which may be conservatively substituted one for the other. In addition, asparagine may be conservatively substituted for glutamine, and vice versa, since both amino acids are amides of dicarboxylic amino acids. In addition, aspartic acid (aspartate) may be conservatively substituted for glutamic acid (glutamate) as both are acidic, charged (hydrophilic) amino acids. In addition, lysine, arginine, and histidine may be conservatively substituted one for the other since each is a basic, charged (hydrophilic) amino acid. Guidance in determining which and how many amino acid residues may be substituted, inserted or deleted without abolishing biological and/or immunological activity may be found using computer programs well known in the art, for example, DNASTAR software. In one embodiment, the sequence of the variant has at least 95% identity, at least 90% identity, at least 85% identity, at least 80% identity, at least 75% identity, at least 70% identity, and/or at least 65% identity with the sequence of the protein in issue.

Reference herein to any specifically named nucleotide sequence (such as a sequence encoding Liver X Receptor, Retinoid X Receptor, etc.) includes within its scope any and all equivalent fragments, homologs, and sequences that hybridize under highly stringent and/or medium stringent conditions to the specifically named nucleotide sequence, and that have at least one of the biological activities (such as those disclosed herein and/or known in the art) of the specifically named nucleotide sequence, wherein the biological activity is detectable by any method.

The “fragment” or “portion” may range in size from an exemplary 5, 10, 20, 50, or 100 contiguous nucleotide residues to the entire nucleic acid sequence minus one nucleic acid residue. Thus, a nucleic acid sequence comprising “at least a portion of” a nucleotide sequence (such as sequences encoding Liver X Receptor, Retinoid X Receptor, etc.) comprises from five (5) contiguous nucleotide residues of the nucleotide sequence to the entire nucleotide sequence.

The term “homolog” of a specifically named nucleotide sequence refers to an oligonucleotide sequence, which exhibits greater than 50% identity to the specifically named nucleotide sequence (such as a sequence encoding Liver X Receptor, Retinoid X Receptor, etc). Alternatively, or in addition, a homolog of a specifically named nucleotide sequence is defined as an oligonucleotide sequence which has at least 95% identity, at least 90% identity, at least 85% identity, at least 80% identity, at least 75% identity, at least 70% identity, and/or at least 65% identity to nucleotide sequence in issue.

With respect to sequences that hybridize under stringent conditions to the specifically named nucleotide sequence (such as a sequence encoding Liver X Receptor, Retinoid X Receptor, etc), high stringency conditions comprise conditions equivalent to binding or hybridization at 68° C. in a solution containing 5×SSPE, 1% SDS, 5×Denhardt's reagent and 100 μg/ml denatured salmon sperm DNA followed by washing in a solution containing 0.1×SSPE, and 0.1% SDS at 68° C. “Medium stringency conditions” when used in reference to nucleic acid hybridization comprise conditions equivalent to binding or hybridization at 42° C. in a solution of 5×SSPE (43.8 g/l NaCl, 6.9 g/l NaH₂PO₄—H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 85% to 95% homology to the first nucleic acid sequence, then another hybridization condition is the to be equivalent to the hybridization condition of interest if this other hybridization condition also results in hybridization of the first nucleic acid sequence with the other nucleic acid sequences that have from 85% to 95% homology to the first nucleic acid sequence.

As will be understood by those of skill in the art, it may be advantageous to produce a nucleotide sequence encoding a protein of interest, wherein the nucleotide sequence possesses non-naturally occurring codons. Therefore, in some embodiments, codons preferred by a particular prokaryotic or eukaryotic host (Murray et al. (1989) Nucl Acids Res., 17) are selected, for example, to increase the rate of expression or to produce recombinant RNA transcripts having desirable properties, such as a longer half-life, than transcripts produced from naturally occurring sequence.

A “composition” comprising a particular polynucleotide sequence (such as a sequence encoding Liver X Receptor, Liver X Receptor agonist, Retinoid X Receptor, Retinoid X Receptor agonist, etc.) and/or comprising a particular protein sequence (such as Liver X Receptor, Liver X Receptor agonist, Retinoid X Receptor, Retinoid X Receptor agonist, etc.) as used herein refers broadly to any composition containing the recited polynucleotide sequence (and/or its equivalent fragments, homologs, and sequences that hybridize under highly stringent and/or medium stringent conditions to the specifically named nucleotide sequence) and/or the recited protein sequence (and/or its equivalent fragments, fusion proteins, and variants), respectively. The composition may comprise an aqueous solution containing, for example, salts (e.g., NaCl), detergents (e.g., SDS), and other components (e.g., Denhardt's solution, dry milk, salmon sperm DNA, etc.).

The terms nucleotide sequence “comprising a particular nucleic acid sequence” and protein “comprising a particular amino acid sequence” and equivalents of these terms, refer to any nucleotide sequence of interest (such as a sequence encoding Liver X Receptor, Liver X Receptor agonist, Retinoid X Receptor, Retinoid X Receptor agonist, etc.) and to any protein of interest (such as Liver X Receptor, Liver X Receptor agonist, Retinoid X Receptor, Retinoid X Receptor agonist, etc.), respectively, that contain the particularly named nucleic acid sequence (and/or its equivalent fragments, homologs, and sequences that hybridize under highly stringent and/or medium stringent conditions to the specifically named nucleotide sequence) and the particularly named amino acid sequence (and/or its equivalent fragments, fusion proteins, and variants), respectively. The invention does not limit the source (e.g., cell type, tissue, animal, etc.), nature (e.g., synthetic, recombinant, purified from cell extract, etc.), and/or sequence of the nucleotide sequence of interest and/or protein of interest. In one embodiment, the nucleotide sequence of interest and protein of interest include coding sequences of structural genes (e.g., probe genes, reporter genes, selection marker genes, oncogenes, drug resistance genes, growth factors, etc.).

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in cells and animals, as exemplified herein.

The term “target RNA molecule” refers to an RNA molecule to which at least one strand of the short double-stranded region of a siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

### DESCRIPTION OF THE INVENTION

Microbe-macrophage interactions play a central role in the pathogenesis of infections. The ability of some bacterial pathogens to induce macrophage apoptosis was suggested to contribute to their ability to elude innate immune responses and successfully colonize the host. Therefore, the present invention relates to microbial infection, and in particular, the reduction of apoptosis associated with microbial infection wherein activation of Liver X Receptors (LXRs) and Retinoid X Receptors (RXRs) inhibits apoptotic responses of macrophages (such as when macrophages are exposed to inducers of apoptosis, experience M-CSF withdrawal in culture, etc.). The present invention also relates to the screening of Liver X Receptor and Retinoid X Receptor agonists that reduce apoptosis, and the treatment and analysis of microbial infection in vivo. In one embodiment, the present invention relates to Liver X Receptor and Retinoid X Receptor agonists including but not limited to those that reduce the activity of pro-apoptotic gene(s). In another embodiment, the present invention relates to Liver X Receptor and Retinoid X Receptor agonists including but not limited to those that increase the activity of anti-apoptotic gene(s). In one embodiment, the present invention relates to agents including but not limited to those agents capable of increasing the activity of Liver X Receptor and/or Retinoid X Receptor. The invention further provides methods for treating and/or analyzing microbial infections in cells, tissues, animals, and the like. The methods of the invention are useful in, for example, the diagnosis, prophylaxis, and reduction of symptoms of diseases and conditions that are associated with microbial infections including multiple infections (e.g., bacterial and viral infections). The methods of the present invention are also useful in identifying treatment agents, and in determining the mechanisms that underlie interactions of Liver X Receptor and/or Retinoid X Receptor, their agonists, and cellular apoptosis.

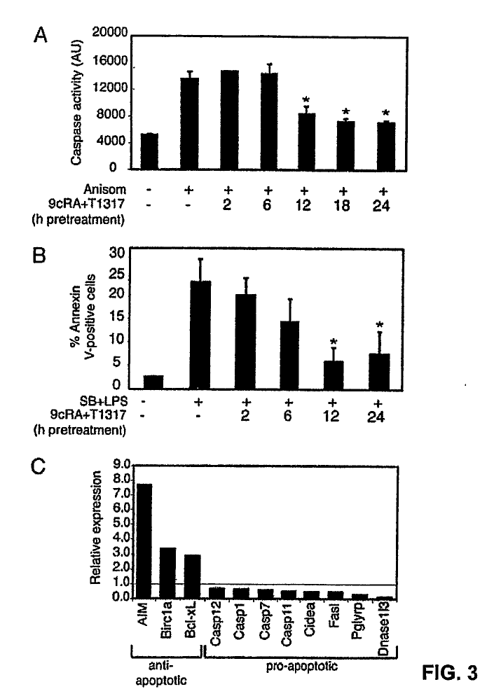

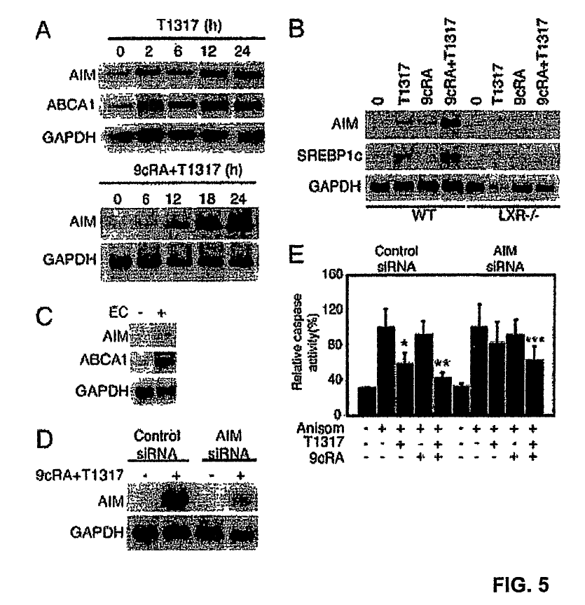

In one embodiment, the agent that increases activity of Liver X Receptor and/or Retinoid X Receptor alters activity of an apoptotic regulator protein. However, the present invention is not limited to alteration of an apoptotic regulator protein. Indeed, other factors may also be regulated, including, but not limited to such molecules as anti-apoptotic molecules, for example, AIM, also known as CT-2/Api6; (Maxwell et al. (2003) J Lipid Res 44, 2109-19); Birc1β (also known as Neuro AIP1), BC1-X_{L}, ABCA1, and the like. In one embodiment, LXR and/or RXR agonists induce the expression of anti-apoptotic regulators, for example, AIM/CT2, Bcl-X_{L}, and Birc1β (see expression profiling studies demonstrating such increase in expression shown in FIG. 3c). In another embodiment, LXR and/or RXR agonists inhibit the expression of pro-apoptotic molecules, for example, TLR4, Bcl2, Bag3 and Birc1a. In one embodiment, reducing activity of an anti-apoptotic regulator protein reduces Liver X Receptor and/or Retinoid X Receptor activity, see, for example, AIM in FIG. 5c. In one embodiment, LXR activation inhibited LPS-dependent induction of the pro-apoptotic factors Bax, Bak, Bcl211, and caspases 1, 3, 4/11, 7, 8 and 12.

In one embodiment, the agent that increases activity of Liver X Receptor and/or Retinoid X Receptor is an agent that reduces pro-apoptotic factors Bax, Bak, Bcl211, and caspases 1, 3, 4/11, 7, 8 and 12. In one embodiment, the agent that increases activity of Liver X Receptor and/or Retinoid X Receptor is a peptide, such as a peptide that interferes with apoptotic activity.

In one preferred embodiment, the agent that increases activity of Liver X Receptor and/or Retinoid X Receptor (LXR and/or RXR), is an antibody, such as LXR or RXR peptide antibody, and/or LXR or RXR sequence antibody. The terms “antibody” and “immunoglobulin” are interchangeably used to refer to a glycoprotein or a portion thereof (including single chain antibodies), which is evoked in an animal by an immunogen and which demonstrates specificity to the immunogen, or, more specifically, to one or more epitopes contained in the immunogen. The term “antibody” includes polyclonal antibodies, monoclonal antibodies, naturally occurring antibodies as well as non-naturally occurring antibodies, including, for example, single chain antibodies, chimeric, bifunctional and humanized antibodies, as well as antigen-binding fragments thereof, including, for example, Fab, F(ab′)2, Fab fragments, Fd fragments, and Ev fragments of an antibody, as well as a Fab expression library. It is intended that the term “antibody” encompass any immunoglobulin (e.g., IgG, IgM, IgA, IgE, IgD, etc.) obtained from any source (e.g., humans, rodents, non-human primates, caprines, bovines, equines, ovines, etc.). The term “polyclonal antibody” refers to an immunoglobulin produced from more than a single clone of plasma cells; in contrast “monoclonal antibody” refers to an immunoglobulin produced from a single clone of plasma cells. Monoclonal and polyclonal antibodies may or may not be purified. For example, polyclonal antibodies contained in crude antiserum may be used in this unpurified state.

Naturally occurring antibodies may be generated in any species including murine, rat, rabbit, hamster, human, and simian species using methods known in the art. Non-naturally occurring antibodies can be constructed using solid phase peptide synthesis, can be produced recombinantly or can be obtained, for example, by screening combinatorial libraries consisting of variable heavy chains and variable light chains as previously described (Huse et al. Science 246:1275-1281, 1989). These and other methods of making, for example, chimeric, humanized, CDR-grafted, single chain, and bifunctional antibodies are well known to those skilled in the art (Winter and Harris (1993) Immunol Today 14:243-246; Ward et al. (1989) Nature 341:544-546; Hilyard et al. Protein Engineering: A practical approach (IRL Press 1992); and Borrabeck, Antibody Engineering, 2d ed. (Oxford University Press 1995)).

Those skilled in the art know how to make polyclonal and monoclonal antibodies, which are specific to a desirable polypeptide. For the production of monoclonal and polyclonal antibodies, various host animals can be immunized by injection with the peptide corresponding to any molecule of interest in the present invention, including but not limited to rabbits, mice, rats, sheep, goats, chickens, etc. In one preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies directed toward molecules of interest in the present invention, any technique that provides for the production of antibody molecules by continuous cell lines in culture may be used (See, e.g., Harlow and Lane, Antibodies: A Laboratory Manual, Cold Spring Harbor Laboratory Press, Cold Spring Harbor, N.Y.). These include but are not limited to the hybridoma technique originally developed by Köhler and Milstein (Köhler and Milstein, Nature 256:495-497, 1975), as well as the trioma technique, the human B-cell hybridoma technique (See e.g., Kozbor et al. Immunol. Today 4:72, 1983), and the EBV-hybridoma technique to produce human monoclonal antibodies (Cole et al., in Monoclonal Antibodies and Cancer Therapy, Alan R. Liss, Inc., pp. 77-96, 1985). In some particularly preferred embodiments of the present invention, the present invention provides monoclonal antibodies of the IgG class.

In additional embodiments of the invention, monoclonal antibodies can be produced in germ-free animals utilizing technology such as that described in PCT/US90/02545. In addition, human antibodies may be used and can be obtained by using human hybridomas (Cote et al. Proc. Natl. Acad. Sci. U.S.A. 80:2026-2030, 1983) or by transforming human B cells with EBV virus in vitro (Cole et al. in Monoclonal Antibodies and Cancer Therapy, Alan R. Liss, pp. 77-96, 1985).

Furthermore, techniques described for the production of single chain antibodies (See e.g., U.S. Pat. No. 4,946,778; herein incorporated by reference) can be adapted to produce single chain antibodies that specifically recognize a molecule of interest (e.g., at least a portion of an AUBP or mammalian exosome, as described herein). An additional embodiment of the invention utilizes the techniques described for the construction of Fab expression libraries (Huse et al. Science 246:1275-1281, 1989) to allow rapid and easy identification of monoclonal Fab fragments with the desired specificity for a particular protein or epitope of interest (e.g., at least a portion of an AUBP or mammalian exosome).

The invention also contemplates humanized antibodies. Humanized antibodies may be generated using methods known in the art, including those described in U.S. Pat. Nos. 5,545,806; 5,569,825 and 5,625,126, the entire contents of which are incorporated by reference. Such methods include, for example, generation of transgenic non-human animals which contain human immunoglobulin chain genes and which are capable of expressing these genes to produce a repertoire of antibodies of various isotypes encoded by the human immunoglobulin genes.

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al. Science, 246:1275-1281, 1989) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

In the production of antibodies, screening for the desired antibody can be accomplished by techniques known in the art (e.g., radioimmunoassay, ELISA [enzyme-linked immunosorbent assay], “sandwich” immunoassays, immunoradiometric assays, gel diffusion precipitin reactions, immunodiffusion assays, in situ immunoassays [e.g., using colloidal gold, enzyme or radioisotope labels], Western blots, precipitation reactions, agglutination assays (e.g., gel agglutination assays, hemagglutination assays, etc.), complement fixation assays, immunofluorescence assays, protein A assays, and immiunoelectrophoresis assays, etc.

In an alternative embodiment, the agent that alters the level of binding of LXR and/or RXR with a LXR ligand and/or a RXR ligand sequence, respectively, is a nucleic acid sequence. The terms nucleic acid sequence therein refer to two or more nucleotides, which are covalently linked to each other. Included within this definition are oligonucleotides, polynucleotide, and fragments or portions thereof, DNA or RNA of genomic or synthetic origin, which may be single- or double-stranded, and represent the sense or antisense strand. Nucleic acid sequences, which are particularly useful in the instant invention, include, without limitation, antisense sequences and ribozymes. In an example herein incorporated by reference, Flavell et al. Aug. 21, 2003 U.S. Patent Appln No, 20030157539A1, a nucleic acid inhibitor comprising IRAK-M reduces toll-like receptor signaling.

In one embodiment, the agent that alters the level of LXR and/or RXR is an antisense nucleic acid sequence. Antisense sequences have been successfully used to inhibit the expression of several genes (Markus-Sekura, Anal. Biochem. 172:289-295, 1988; Hambor et al. J. Exp. Med. 168:1237-1245, 1988; and patent EP140308, incorporated in its entirety by reference) including the gene encoding VCAM1, one of the integrin α-4/β-1 ligands (U.S. Pat. No. 6,252,043, incorporated in its entirety by reference). The terms “antisense DNA sequence” and “antisense sequence” as used herein interchangeably refer to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex, which is transcribed by a cell in its natural state into a “sense mRNA.” Sense mRNA generally is ultimately translated into a polypeptide. Thus, an “antisense DNA sequence” is a sequence which has the same sequence as the non-coding strand in a DNA duplex, and which encodes an “antisense RNA” (i.e., a ribonucleotide sequence whose sequence is complementary to a “sense mRNA” sequence). The designation (−) (i.e., “negative”) is sometimes used in reference to the antisense strand, with the designation (+) sometimes used in reference to the sense (i.e., “positive”) strand. Antisense RNA may be produced by any method, including synthesis by splicing an antisense DNA sequence to a promoter, which permits the synthesis of antisense RNA. The transcribed antisense RNA strand combines with natural mRNA produced by the cell to form duplexes. These duplexes then either block the further transcription of the mRNA or its translation, or promote its degradation.

Antisense oligonucleotide sequences may be synthesized using any of a number of methods known in the art (such as solid support and commercially available DNA synthesizers, standard phosphoramidate chemistry techniques, and commercially available services, e.g., Genta, Inc.).

In some alternative embodiments, the agent that alters the level of LXR and/or RXR sequence is a ribozyme nucleic acid sequence, for example, a ribozyme, a hammerhead ribozyme, Inozyme, Zinzyme, G-cleaver, Amberzyme, or DNAzyme, and the like, herein incorporated by reference as described in U.S. Patent Appln. No., 20030119017A1, McSwiggen, Jun. 26, 2003. Ribozyme sequences have been successfully used to inhibit the expression of several genes including the gene encoding VCAM1, which is one of the integrin α-4/β-1 ligands (U.S. Pat. No. 6,252,043, incorporated in its entirety by reference). The term “ribozyme” refers to an RNA sequence that hybridizes to a complementary sequence in a substrate RNA and cleaves the substrate RNA in a sequence specific manner at a substrate cleavage site. Typically, a ribozyme contains a “catalytic region” flanked by two “binding regions.” The ribozyme binding regions hybridize to the substrate RNA, while the catalytic region cleaves the substrate RNA at a “substrate cleavage site” to yield a “cleaved RNA product.” Examples of ribosomes that modulate genes related to apoptosis are NF-Kappaβ genes, such as REL-A, REL-B, REL (c-rel), NFKB1 (p105/p50) and NFKB2 (p100)/p52/p49), herein incorporated by reference, are demonstrate in U.S. Patent Appln No., 20020177568A1, Stinchcomb, et al. Nov. 28, 2002. Further types of nucleic acid molecules used to modulate other types of apoptotic molecules including PKR and IKK genes, herein incorporated by reference, are demonstrated in U.S. Patent Appln. No., 20030119017A1, McSwiggen, et al. Jun. 26, 2003.

Molecules which find use as agents for specifically altering the level of specific binding of LXR and/or RXR with effector molecule sequences include organic molecules, inorganic molecules, and libraries of any type of molecule, which can be screened using a method of the invention, and which may be prepared using methods known in the art. These agents are made by methods for preparing oligonucleotide libraries (Gold et al. U.S. Pat. No. 5,270,163, herein incorporated by reference); peptide libraries (Koivunen et al. J. Cell Biol., 124: 373-380, 1994); peptidomimetic libraries (Blondelle et al. Trends Anal. Chem. 14:83-92, 1995); oligosaccharide libraries (York et al. Carb. Res. 285:99-128, 1996; Liang et al. Science 274:1520-1522, 1996; and Ding et al. Adv. Expt. Med. Biol. 376:261-269, 1995); lipoprotein libraries (de Kruif et al. FEBS Lett., 399:232-236, 1996); glycoprotein or glycolipid libraries (Karaoglu et al. J. Cell Biol. 130:567-577, 1995); or chemical libraries containing, for example, drugs or other pharmaceutical agents (Gordon et al. J. Med. Chem. 37:1385-1401, 1994; Ecker and Crook, Bio/Technology 13:351-360, 1995; U.S. Pat. No. 5,760,029, herein incorporated by reference). Libraries of diverse molecules also can be obtained from commercial sources.

Macrophages are pivotal effector cells of the innate immune system, vital for recognition and elimination of microbial pathogens (Aderem et al. Nature 406, 782-7, 2000). As used herein, the term “macrophage” and “macrophage cells” refers to a phagocytic cell of the myeloid lineage in the mononuclear phagocyte system (a system comprising blood monocytes and tissue macrophages). Macrophages can derive from myeloid precursors such as those found in the bone marrow and thus share characteristics such as cell surface markers with many other myeloid derived cells (e.g., human macrophages can express numerous markers such as CD11b, CD11c, CD16, CD68, CD14, CD80, CD86, HLA-DR and the like that are shared with other myeloid precursors; similarly, mouse macrophages can share markers such as Mac-1, F4/80, and the like; however when these markers are used in certain combinations; including qualitative and quantitative measurements, they can also be used to distinguish between macrophages and other cells of similar myeloid origins, maturation stages, activation levels and functional characteristics, for example, in mouse, see, Inaba et al., PNAS 90(7):3038-42, 1993; in human see). As a further example, macrophages and dendritic cells are derived from similar primordial cells and thus share many characteristics with each other including identifying markers, capacity for becoming “activated” in response to antigens, phagocytic functions and the like, for the greater purpose of responding to stimuli requiring a particular response. Macrophages and DCs are so closely related that CD34+ precursors in normal human bone marrow (BM) can be selectively cultured to generate populations of macrophages or DCs or mixed cultures of both (Szabolcs et al. Blood. June 1; 87(11):4520-30, 1996; Szabolcs et al. J Leukoc Biol. 1999 August; 66(2):205-8). Further, monocytes are known to develop into dendritic cells (DCs) that migrate to lymph nodes (LNs) and present antigens to T cells (see Chapts. 15-16, Fundamental Immunology Ed., Paul, Fifth Edition, September 2003). Macrophages are found throughout an organism in various stages of maturation and activation (e.g. monocytes, macrophages, activated macrophages, cytokine and/or chemokine activated macrophages (also referred to as Activated Killer Monocytes) and the like). Macrophages have a variety of morphological forms, phenotypes and functions, sometimes referred to as subpopulations, suited for residing within each type of tissue (e.g. Kupffer cells in the liver, alveolar macrophages in the lungs, microglial in the brain, macrophages in the thymic cortex, macrophages in the marginal zone of the spleen, macrophages in peripheral areas of granulomas, and the like). Macrophages have different stages of attachment ranging from non-attached (e.g. suspension, free floating, monocytes in early stages of culture, and the like) as when circulating within the blood stream, to various intermediate stages of attachment (when migrating into and out of endothelium, in cell cultures and the like) and attached (e.g. within specific tissues, attached cultures and the like). Macrophages display a range of functional activities depending upon their maturation stage, activation state, tissue location, and attachment level. It is not intended that the present invention be limited to a particular function or phenotype or maturation stage of macrophage cells. In one embodiment, macrophages are cultured from bone marrow cells (e.g. Valledor et al. (1999) J Immunol 163, 2452-62). In one embodiment, the macrophage cells are activated macrophages (for example mature macrophages, infected macrophages, cultured macrophages, cytokine induced macrophage, lymphocyte activated macrophages and the like). In one embodiment macrophage cells are phagocytic. In one embodiment macrophage cells contain numerous granules of bactericidal molecules. In one embodiment, the macrophage cells are monocytes (for example immature macrophages, and the like). In yet another embodiment, macrophages are immunocytes of myeloid lineage (for example, dendritic cells, myeloid dendritic cells and the like). In another embodiment, the macrophage cells are immunocytes functionally equivalent to macrophages (for example, Kupffer cells, microglia, astrocytes, and the like). In another embodiment, macrophage cells are immunocytes of lymphoid origin (for example, splenic cells, lymphoid derived dendritic cells, and the like). In one embodiment, macrophages are precursors to dendritic cells (Rotta et al. (2003) J Exp Med. 198:1253-63). However they globally function as phagocytes that ingest microbes and particles for destruction and particularly in triggering microbial immune responses. Macrophages can trigger immune responses by presenting microbial antigens to immunocompetent cells while in an activated state. Many factors contribute to activating macrophages including microbial infection wherein the microbe is killed and degraded within the phagosome, cytokines and chemokines are being produced to recruit lymphoid cells and other types of leukocytes to sites of infection, and components of the pathogen are presented to T cells, resulting in adaptive immunity (Aderem et al. (2000) Nature 406:782-7).

It is not intended that the present invention be limited to a particular source of macrophage cells. In one embodiment, macrophage cells are derived from bone-marrrow cells (BMDM). In one embodiment, macrophage cells are derived from fetal-liver (FLDMs). In one embodiment, macrophage cells are located within an animal. In one embodiment, macrophages are located within the red pulp area of spleens.

It is not intended that the present invention be limited to a particular stage of development of the macrophage cell host. In one embodiment, macrophages cells are derived from mature (adult) animals. In one embodiment, macrophage cells are derived from 8-10 week-old mice.

In one embodiment, the macrophage cells are activated macrophages (for example, infected macrophages, mature macrophages, cultured macrophages, cytokine induced macrophages, lymphocyte activated macrophages and the like). In one embodiment macrophage cells are phagocytic. In one embodiment macrophage cells contain numerous granules of bactericidal molecules. In one embodiment, the macrophage cells are monocytes (for example, immature macrophages, and the like). In yet another embodiment, macrophages are immunocytes of macrophage lineage (for example, dendritic cells, Langerhans cells, dermal dendritic cells and the like). In another embodiment, the macrophage cells are immunocytes functionally equivalent to macrophages (for example, Kupffer cells, microglia, astrocytes, and the like). In another embodiment, macrophage cells are immunocytes of lymphoid origin (for example, lymphoid derived dendritic cells, and the like).

### DETAILED DESCRIPTION OF THE INVENTION

### I. Macrophage Function and Expression of Nuclear Receptors

Macrophages serve essential functions as regulators of immunity and homeostasis (Celada et al. (1994) Immunol Today 15, 100-2; and Gordon (1998) Res Immunol 149, 685-8). As participants in native immunity, macrophages phagocytose and kill invading microorganisms and elaborate signaling molecules that amplify acute inflammatory responses. Macrophages also contribute to acquired immune responses via specialized functions that include antigen presentation and regulation of T cell responses. Regulation of macrophage differentiation and survival is thus critical to the overall control of the magnitude, duration and characteristics of immune responses. Programmed cell death, or apoptosis, of lymphocyte and myeloid cells is tightly regulated through cell death receptor and mitochondrial pathways to limit amplification of immune responses and facilitate resolution of inflammation (Savill (1997) J. Leukocyte Biol. 61, 375-380). Apoptosis and survival pathways are also targeted by pathogens as a means of either escaping immune surveillance or establishing residence within host cells (Weinrauch et al. (1999) Annu Rev Microbiol 53, 155-87). The inhibition of macrophage apoptosis is a desirable strategy for augmenting innate immunity to highly virulent bacterial pathogens, such as Bacillus anthracis, Yersinia pestis, Salmonella spp. and Shigella flexneri, that have evolved various ways to kill host macrophages. The execution of all forms of programmed cell death involves the proteolytic activation of a cascade of intracellular cysteine proteases known as caspases. Downstream effector caspases cleave specific protein targets and mediate the deliberate disassembly of the cell into apoptotic bodies (Cohen (1997) Biochem J 326, 1-16). A number of regulators of apoptosis function upstream and downstream of caspases by either promoting or suppressing their protease activities. For example, anti-apoptotic members of the Bcl2 family act, at least in part, to preserve mitochondrial integrity and function, including its transmembrane potential, calcium buffering capacity, respiration efficiency and prevent the release of pro-apoptotic components. Other members of the Bcl2 family have an opposite effect and mediate mitochondrial dysfunction and eventual release of pro-apoptotic mediators (reviewed in Ranger et al. (2001) Nat Genet. 28, 113-8). One approach of the present invention for inhibition of macrophage apoptosis involves the manipulation of the expression of such proteins.

Nuclear receptors are ligand-dependent transcription factors that regulate diverse aspects of development and homeostasis (Mangelsdorf et al. (1995) Cell 83, 835-839). Several members of this family influence immune responses by activating or repressing cell-specific programs of gene expression in myeloid and/or lymphoid cells (Welch et al. (2003) in The Macrophage As A Therapeutic Target, ed. Gordon, S. (Springer, Berlin), Vol. 158, pp. 209-226). For example, the glucocorticoid receptor exerts potent anti-inflammatory effects in part through its ability to inhibit the actions of pro-inflammatory transcription factors, such as AP-1 and NF-κB, and induce apoptosis of lymphocytes (Karin (1998) Cell 93, 487-490; and De Bosscher et al. (2003) Endocr Rev 24, 488-522). Liver X receptors (LXRs) represent a subset of the nuclear receptor superfamily that are regulated by oxidized forms of cholesterol (oxysterols) and intermediate products of the cholesterol biosynthetic pathway (Janowski et al. (1996) Nature 383, 728-731; and Janowski et al. (1999) Proc Natl Acad Sci USA 96, 266-71). Two LXR isoforms have been identified, LXRα (NR1H3) and β (NR1H2), which are encoded by distinct genes. LXRs form obligate heterodimers with retinoid X receptors (RXR), which are themselves members of the nuclear receptor superfamily that can be regulated by 9-cis retinoic acid (9cRA) and long chain polyunsaturated fatty acids (Heyman et al. (1992) Cell 68, 397-406; Bourguet et al. (2000) Molecular Cell 5, 289-298; and Mata de Urquiza et al. (2000) Science 290, 2140-4). LXR-RXR heterodimers regulate their target genes by recognizing specific LXR response elements consisting of two direct hexanucleotide repeats separated by four nucleotides (Willy et al. (1995) Genes Dev 9, 1033-45). Without ligands, LXR/RXR heterodimers actively repress transcription of target genes through recruitment of the nuclear receptor corepressors NCoR and SMRT (Wagner et al. (2003) Mol Cell Biol 23, 5780-9; and Hu et al. (2003) Mol Endocrinol 17, 1019-26). Upon binding either LXR or RXR ligands, corepressors are exchanged with nuclear receptor coactivators, resulting in transcriptional activation. LXR/RXR heterodimers induce expression of genes that mediate cholesterol efflux from cells and its ultimate excretion into bile (Repa et al. (1999) Curr Opin Biotecbnol 10, 557-63). This activity has been shown to be important in the regulation of cholesterol homeostasis in macrophages, which can accumulate massive amounts of cholesterol in disease settings, such as atherosclerosis. Recent studies have also demonstrated that LXRs inhibit transcriptional responses to activation of Toll-like receptor 4 (TLR4) in macrophages by antagonizing the actions of NF-κB transcription factors (Joseph et al. (2003) Nat Med 9, 213-9). Recently, LXR-null macrophages were observed to undergo accelerated apoptosis when challenged with Listeria mollocytogenes, and to exhibit defective bacterial clearance in vivo (Joseph et al., (2004) Cell 119, 299-309). Here the inventors have significantly extended these studies by providing evidence that LXRs and RXRs regulate macrophage survival, indicating that they are important modulators of innate immunity.

### II. Roles of LXRs in the Control of Macrophage Proliferation and Survival

LXRs play critical roles in the regulation of cholesterol and fatty acid homeostasis (Repa and Mangelsdorf (2000) Annu Rev Cell Dev Biol 16, 459-81). In macrophages, LXRs activate the expression of a set of genes, such as the ABCA1 cholesterol transporter, that act to reduce cellular cholesterol levels (Venkateswaran et al. (2000) Proc Natl Acad Sci USA 97, 12097-102; and Repa et al. (2000) Science 289, 1524-9). This function of LXRs has been most intensively studied in the context of atherosclerosis, a disease in which cholesterol-loaded macrophages accumulate within the walls of large arteries (Ricote et al. (2004) Arterioscler Thromb Vasc Biol, 24, 230-239). Recent studies demonstrating that synthetic LXR agonists can also inhibit transcriptional events induced by TLR4 signaling suggest that LXRs have additional roles in the regulation of immune responses (Joseph et al. (2003) Nat Med 9, 213-9). The present invention extends this observation by providing compositions and methods for activating LXRs to promote macrophage survival.

The inventors contemplate that one important anti-apoptotic role of LXRs is the protection of macrophages from cholesterol toxicity due to phagocytosis of dead cells. Programmed cell death is an important phenomenon during resolution of inflammation and oxidative damage is a component of the apoptotic program (Buttke et al. (1994) Immunol Today 15, 7-10). The resolution of acute inflammation requires bulk clearance of infiltrating inflammatory cells in an ordered manner. Neutrophils participate in early phases of the inflammatory process by phagocytosing and destroying the agents that cause inflammation. Rapidly after their activation, they undergo apoptosis (Bellingan et al. (1996) J Immunol 157, 2577-85). Resident macrophages play an essential role in clearance of apoptotic bodies and debris generated during those conditions and the uptake of apoptotic cells results in a significant load of cellular cholesterol. Conversion of a fraction of this excess cholesterol to oxysterol ligands for LXR is contemplated to result in activation of genes such as ABCA1 required for cholesterol efflux.

Unexpectedly, as shown herein for the first time, activation of LXR and RXR protects macrophages from apoptotic signaling pathways that are stimulated by bacterial pathogens including B. anthracis and S. typhimurium. Some pathogens, such as Listeria and Legionella, can reside intracellularly within macrophages, and thereby elude immune clearance (Navarre et al. (2000) Cell Microbiol 2, 265-73). In contrast, other pathogens, exemplified by Salmonella, Shigella and Yersinia, induce macrophage apoptosis and stimulate the release of proinflammatory cytokines (Navarre et al. (2000) Cell Microbiol 2, 265-73). The present invention demonstrates that LXR and RXR agonists are suitable for treating microbial infections and as tools for investigating the importance of apoptosis in the pathogenicity of various bacterial infections in vivo.

Activation of LXR predominantly antagonized the apoptotic program induced by engagement of TLR4 by both positively and negatively regulating gene expression. Furthermore, the combination of LXR and RXR agonists was more effective at inhibiting macrophage apoptosis than either agonist alone. The anti-apoptotic factors Bcl-X_{L}, Birc1a/NAIP, and AIM/CT2/Api6 were significantly upregulated by the combination of LXR and RXR agonists, suggesting that they are directly or indirectly regulated by RXR/LXR heterodimers. Bcl-X_{L} is an anti-apoptotic form of Bcl-X that is related in structure and function to Bcl-2 (Chao et al. (1995) J Exp Med 182, 821-8). Members of the Bcl-2 family control apoptosis by several mechanisms, including alterations in cytochrome C release, which ultimately regulates caspase activation (Akgul et al. (2001) FEBS Lett 487, 318-22; and Kroemer et al. (1997) Nat Med 3, 614-20). The balance between pro-apoptotic members (e.g., Bax, Bad, and Bak) and anti-apoptotic members (e.g., Bcl-2, BC1-X_{L}, and Mcl-1) determines the fate of many types of cells. Birc1a/NAIP is related to baculoviral inhibitor of apoptosis proteins (IAPs) (Roy et al. (1995) Cell 80, 167-78) and directly inhibits the enzymatic activities of effector caspases 3 and 7 (Maier et al. (2002) J Neurosci 22, 2035-43). In combination with down-regulation of caspases 1, 4/11, 7 and 12, coordinate up-regulation of Bcl-X_{L} and Birc1a/NAIP is contemplated to account for at least some of the ability of LXR and RXR agonists to decrease caspase activities in response to exposure to apoptotic stimuli and bacterial pathogens. AIM/CT2/Api6 was synergistically activated by LXR/RXR agonists and contributed to their anti-apoptotic effects. While the mechanisms responsible for the anti-apoptotic activities of AIM/CT2/Api6 remain to be established, in situ hybridization studies showed high expression in specific macrophage subpopulations, including subsets of Kupffer cells in the liver, macrophages in the thymic cortex, in the marginal zone of the spleen and in peripheral areas of granulomas (Miyazaki et al. (1999) J Exp Med 189, 413-22).

Nuclear receptors also play important physiological roles by negatively regulating gene expression and microarray experiments indicated that LXR/RXR agonists inhibited the expression of several positive regulators and effectors of apoptosis. Mechanisms of negative regulation by nuclear receptors are generally less well understood than those responsible for positive regulation and it is possible that additive/synergistic effects of LXR and RXR agonists results from independent activities of the two receptor subtypes. However, microarray experiments indicated that 9cRA alone had very little inhibitory activity on LPS-dependent gene expression in macrophages. It thus appears that the predominant role of RXR agonists as inhibitors of apoptosis is to potentiate both the positive and negative transcriptional activities of LXR agonists, most likely acting through LXR/RXR heterodimers. Caspases 1, 4/11, 7 and 12 were modestly downregulated (from 1.5 to 2-fold, FIG. 3c, 4d), contributing to reduced caspase activity observed after treatment with LXR/RXR agonists. Intriguingly, the combination of LXR and RXR agonists downregulated several genes that contribute to apoptosis-induced DNA fragmentation. DNase γ and Cidea, which contribute to DNA fragmentation during apoptosis (Shiokawa et al. (2002) J Biol Chem 277, 31031-7; and Inohara et al. (1998) Embo J 17, 2526-33), were strongly downregulated in response to LXR/RXR agonists. LXR/RXR agonists also inhibited the expression of peptidoglycan recognition protein (PGLYP), which forms a cytotoxic complex with heat shock protein 70 (Sashchenko et al. (2004) J Biol Chem 279, 2117-24). In concert, these studies demonstrate that LXR and RXR coordinately regulate the network of genes that control programmed cell death, resulting in protection of macrophages from bacteria-induced apoptosis.

The above description is not intended to convey that any of these cells, proteins, molecules and receptors have only one function. Physiological pathways are in flux, for example apoptotic pathways, and not usually isolated from each other. There are several apoptotic pathways leading towards apoptotic death that overlap with several other pathways leading towards cell survival and proliferation.

### III. Exemplary Embodiments

In one embodiment, macrophage cells express LXR and/or RXR. In another embodiment, cells contacted by compositions of the present invention are any cells that are LPS-responsive. In another embodiment, the cells are any closely related immunocytes expressing LXR and/or RXR (for example, myeloid cells, white blood cells, undifferentiated immunocytes, immature dendritic cells of lymphoid lineage and the like). In another embodiment, the LXRs and/or RXRs are involved in activation of macrophages and their effector functions, including increasing anti-apoptotic and decreasing pro-apoptotic signaling pathways.

As used herein, the terms “Toll-like receptor,” “TLR,” “pattern recognition receptors,” and “PRRs” refer to molecules of the immune system that are activated by microbes and microbial molecules. In one embodiment, a TLR binds to microbial ligands. In one embodiment, a TLR binds to a PAMP. As used herein, the terms “PAMP” and “pathogen-associated molecular pattern” refers to any molecule expressed by microbial pathogens that contain repetitive motifs “patterns” (e.g., lipopolysaccharide (LPS), peptidoglycan, mannan, and the like). It is not intended that the present invention be limited to a particular PAMP. In one embodiment, a PAMP is a molecule that activates a TLR. In one embodiment, a PAMP is a molecule that activates a TLR-4. In one embodiment, a PAMP is a LPS. In one embodiment, a PAMP is a LPS that activates TLR-4. In one embodiment, a PAMP is lipoteichoic acid (LTA).

Apoptosis was also observed upon pretreatment of myeloid cells with type I interferons (IFN) followed by incubation with LPS (Adler et al., Biochem Biophys Res Commun, 215, 921-7, 1995; Lehner et al. Blood 98, 736-42, 2001). Type I IFNs are produced in response to viral infections and it is well established that such infections, for instance with influenza virus, predispose affected individuals to excess mortality from common microbial pathogens, such as Haemophilus influenzae or Streptococcus pneumoniae (Abrahams et al. Lancet 1, 1-11, 1919; Oxford, Rev Med Virol 10(2):119-33, 2000). As used herein, the term “virus” and “viral” refers to obligate, ultramicroscopic, intracellular parasites incapable of autonomous replication (i.e., replication requires the use of the host cell's machinery). Although such microbes do not induce macrophage apoptosis on their own, it was observed that influenza virus infection can markedly enhance the susceptibility of myeloid cells to bacteria-induced apoptosis (Colamussi et al. Blood 93, 2395-403, 1999). It is contemplated that such an effect contributes to the immunodeficiency that is commonly associated with viral infections (Ray, G. C. Influenza, Respiratory Syncytial Virus, Adenovirus, and Other Respiratory Viruses, ed. K. J., R.), Appleton & Lange, Newwalk, Conn., 1994).

As used herein, “double stranded RNA” and “dsRNA” refer to a double stranded ribonucleotide sequence. Double stranded RNA may be chemically synthesized and/or naturally occurring. For example naturally occurring dsRNA includes dsRNA segments (also referred to as dsRNA portions) that are found in, and may be isolated from, virus infected cells. Examples of synthesized segments are presented herein.

An example of a test agent that reduces apoptosis is an agent that interacts with LXR and/or RXR to reduce the translation of viral RNA. An example of a screen for such an agent is described and incorporated by reference in U.S. Pat. Nos. 6,623,961, 5,738,985, 6,156,496, 6,579,674, 6,667,152 and 6,777,179; U.S. Patent Appln. Nos., 2002160976, 2002160977, 2003144226, 2003144226; and PCT publications WO9423041.

As used herein, the terms “Toll-like receptor-4,” “TLR4,” “TLR4,” “human homologue of Drosophila Toll,” “hToll” refers to equivalent proteins, RNA and DNA having homology (partial or complete) (Medzhitov et al. 1997, Nature. 388: 394-397; and Rock et al. 1997, Proc. Natl. Acad. Sci. USA. 95: 558-592).

The inventors demonstrate that macrophage apoptosis by either gram-positive (B. anthracis) or gram-negative (Yersinia, Salmonella) pathogens requires activation via LXR and/or RXR. It is not intended that the present invention be limited to a particular “bacterium,” portion of bacterium or stage of bacterium lifecycle. In one embodiment, the bacterium is chosen from one or more of infectious bacterium. As used herein, the term “infectious” refers to bacterium that are capable of at least one cell division. In another embodiment the bacterium is selected from one or more of whole, intact, inactivated, dead, lysate, fractionated, secreted molecules, endotoxins, outer cell membrane components, pili parts, cell wall parts, coat parts, glycoproteins, glycolipids, polysaccharides, M protein, external parts, membrane parts, internal parts, peptides, lipids, and nucleic acids. In one embodiment, bacterium is a gram-positive bacterium (e.g. Bacillus anthracis Sterne, and the like) (Welkos et al, J Med Microbiol 51, 821-31, 2002). In one embodiment, bacterium is a gram-negative bacterium (e.g. Yersinia species, Salmonella typhimuriun, H. influenza, and the like). In one embodiment, bacterium is wild-type bacterium (e.g. S. typhimurium strains SL1344 and 14028). Further, it is not intended that the bacterium is limited to wild-type bacterium. In one embodiment, bacterium are mutant bacterium and contain one or more inactive genes (e.g. Yersinia pseudotuberculosis YP26 (YopJ-), Salmonella typhimurium 14028 ssaV (contain mutations in genes that code for components of the SPI2 type III protein secretion system) and Salmonella typhimurium 14028 sipB (contain mutations in SipB), and Salmonella typhimurium SL1344/SipB⁻ (Browne et al, Infect Immun 70, 7126-35, 2002), etc.).

It is not intended that the present invention be limited to a particular method of bacterium culture. In one embodiment, B. anthracis Steme strain (Welkos et al. J Med Microbiol 51, 821-31, 2002) was grown overnight on BHI (brain-heart infusion) agar: a single colony was inoculated into BHI broth and grown with vigorous shaking to an OD600 of 0.4. In one embodiment, heat killed B. anthracis, were prepared by resuspending bacterium in PBS as above and heated to 65° C. for 30 min (Welkos et al. J Med Microbiol 51, 821-31, 2002).

It is not intended that the present invention be limited to a particular method of obtaining bacterium. In one embodiment, Y pseudotuberculosis strains YP126 (wt) and YP26 (YopJ-) (Zhang and Bliska, Infect Immun 71, 1513-9, 2003) were obtained from Dr. J. Bliska (SUNY at Stony Brook, N.Y.).

### EXPERIMENTAL

The following examples serve to illustrate certain preferred embodiments and aspects of the present invention and are not to be construed as limiting the scope thereof. In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade/Celsius).

### Example 1

### Materials And Methods

The following is a description of exemplary materials and methods that were used in subsequent examples.

Reagents: It is not intended to limit the source of reagents. In one embodiment, reagents were obtained by donations (for example, T1317 and GW3965 was donated by X-ceptor Therapeutics, Inc., San Diego, Calif.). In one embodiment, reagents were obtained from commercial sources. Anisomycin of Streptomyces griseolus and SB202190 were purchased from Calbiochem (San Diego, Calif.). Cycloheximide of Staphylococcus griseus, 9 cis-retinoic acid and lipopolysaccharide (LPS) were obtained from Sigma (St. Louis, Mo.). 24(S),25-epoxycholesterol (EC) was purchased from BIOMOL Research Laboratories, Inc. (Plymouth Meeting, Pa.). Small interfering RNA (siRNA) was obtained from Ambion (Austin, Tex.).

Sources of mice: It is not intended to limit the source of mice. In one embodiment, mice were obtained by personal donations (for example, LXR^{-/-} mice were obtained from Drs. David Mangelsdorf and Joyce Repa) and LXRα/β^{-/-} mice (Repa et al. (2000) Genes Dev. 14, 2819-2830) were obtained from Dr. David Mangelsdorf). As used herein, the term “transgenic” when used in reference to a tissue or to a plant refers to a tissue or plant, respectively, which comprises one or more cells that contain a transgene, or whose genome has been altered by the introduction of a transgene. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein. In one embodiment, knockout mice were of the C57BL/6 background, which is resistant to LT-induced necrosis. As used herein, the term “knockout” refers to a deletion, deactivation, or ablation of a gene or deficient gene in a mouse or other laboratory animal or any cells in an animal. When the knockout includes the germ cells, subsequent breeding can create a line of animals that are incapable of or produce significantly less of the gene product. As used herein, the term “transgenic” when used in reference to a cell refers to a cell which contains a transgene, or whose genome has been altered by the introduction of a transgene. Bone Marrow-Derived Macrophages (BMDM) and Infections: It is not intended to limit the source of Bone marrow-derived macrophages (BMDM). In one embodiment, BMDMs were isolated from 8-10 week-old mice as described by Valledor et al. (1999) J Immunol 163, 2452-62, herein incorporated by reference. Briefly, the cells were cultured in DMEM (Dulbecco's Modified Eagle's Medium, Cellgro, Mediatech, Inc., Hemdon, Va.) containing 20% FBS (Fetal Bovine Serum, Hyclone, Logan, Utah) and 30% L-cell (C3H mouse fibroblast) conditioned media as a source of M-CSF (Macrophage Colony Stimulating Factor). In one embodiment, macrophages were obtained as a homogeneous population of adherent cells after 6-8 days of culture. Unless otherwise stated, macrophages were used at <80% confluence. Experiments were performed with the approval of the UCSD (University of California at San Diego) Animal Subject Committee.

Bacterial strains and macrophage infections: Wild-type Salmonella typhimurium strains used were SL1344 and 14028. Salmonella typhimurium 14028 ssaV and sipB contain mutations in genes that code for components of the SPI2 type III protein secretion system and SipB, respectively. In one embodiment, Y. pseudotuberculosis strains YP126 (wild type) and YP26 (YopJ2) were obtained from J. Bliska.

The B. anthracis Sterne strain was grown overnight on BHI (brain-heart infusion) agar. A single colony was inoculated into BHI broth or RPMI medium plus 10% fetal calf serum (FCS) (endotoxin-free) in disposable tubes and grown with vigorous shaking to an OD₆₀₀ of 0.4. Bacteria were washed with PBS and resuspended in PBS. To prepare heat-killed B. anthracis, bacterial suspensions in PBS were heated to 65° C. for 30 minutes. A macrophage culture was infected as indicated and incubated for 1 h at 37° C. in 5% CO₂/95% air. Gentamicin was added to a final concentration of 20 mgml (Diebold et al. Nature 424, 324-328, 2003). After 20 h, the medium was removed and cells were fixed with 4% paraformaldehyde in PBS.

Apoptosis test: It is not intended to limit the type of test for identifying and measuring apoptosis (for example, DNA fragmentation using DNA assays, flow cytometry assays, etc., cell death assays using microscopy, etc., caspase activation, using fluorimetric assays, etc.). In one embodiment, DNA fragmentation was measured by a photometric enzyme immunoassay (Cell Death Detection ELISA Plus, F. Hoffmann-La Roche Ltd, Basel, Switzerland), in triplicate samples, wherein the assay was directed towards the recognition of histone-associated DNA fragments. In some experiments, the measurement of DNA fragmentation was performed by flow cytometry. Briefly, the cells were fixed in 70% ethanol for 30 min at room temperature and then stained with propidium iodide (PI, 30 μg/ml) in 0.25% tryton/PBS containing RNase A. In one embodiment, DNA fragmentation was measured by analyzing the DNA content of 10,000 cells by flow cytometry using an FL-2A channel. In one embodiment, general caspase activation was measured in triplicate samples with a quantitative fluorimetric assay (Homogenous Caspases Assay, fluorimetric, F. Hoffmann-La Roche Ltd). In one embodiment, the progression towards cell death was assayed. Briefly, the apoptotic related exposure of phosphatidylserine in the outer leaflet of the plasma membrane was measured by annexin V staining (Koopman et al. (1994) Blood 84, 1415-20; and Vermes et al. (1995) J Immunol Methods 184, 39-51), and described, supra. Macrophages were plated in slide chambers before exposure to LXR agonists and apoptotic signals. Annexin V-Alexa 568 staining (F. Hoffmann-La Roche Ltd) was performed in situ without detaching the cells from the plate. Hoechst dye was used for nuclear staining. Several fields of at least 120 cells each were counted and the percentage of annexin V-positive cells versus total cells was determined.

Microarray analysis: In one embodiment, large numbers of genes were assayed for relative expression levels using one or more of an Affymetrix U74A array and a Codelink Uniset 1 mouse array. Total RNA was isolated and purified using Trizol reagent (Invitrogen Life Technologies, Carlsbad, Calif.) and RNeasy columns (Qiagen, Valencia, Calif.). cRNA was generated from 10 μg total RNA using Superscript (Invitrogen) and the High Yield RNA transcription labeling kit (Enzo Biochem. Inc., Farmingdale, N.Y.). Duplicate samples of fragmented cRNA were hybridized to Affymetrix U74A arrays or Codelink Uniset 1 mouse arrays according to manufacture's instruction. Data was analyzed with Microarray Suite (Affymetrix, Santa Clara, Calif.) and Genespring software (Silicongenetics, Redwood City, Calif.).

Northern blots: In one embodiment, mRNA for individual gene expression was analyzed. Total RNA was purified using Trizol. RNA samples (10 μg per lane) were separated in 1.2% agarose gels containing formaldehyde and transferred to Genescreen nylon membranes (NEN, Boston, Mass.). Hybridization to labeled probes was performed using Quickhyb (Stratagene, La Jolla, Calif.).

siRNA-mediated knockdown of AIM (Apoptosis Inhibitor expressed by Macrophages): In one embodiment, a siRNA is directed to a target sequence of the AIM transcript. For example, target sequences used were: AIM-1, ⁵′AACGGAAGACACGTTGGCTCA³′ (SEQ ID NO:1); and AIM-2, ⁵′AAGATGTCGTGTTCTGGACAA³′ (SEQ ID NO:2). In one embodiment, a control was a target sequence that is not directed to any known vertebrate gene, for example, a scrambled siRNA was developed from the following target sequence: ⁵′AAGATACTCGTGATTGCACAC³′ (SEQ ID NO:3). In experiments directed to study macrophage apoptosis, 8×10⁴ cells were transfected using Superfect (Qiagen) with 0.4 μM siRNA. The same ratio siRNA/cell numbers was maintained in higher scale experiments.

### Example 2

### LXR and RXR Agonists Inhibit Apoptotic Responses

### To Growth Factor Withdrawal and Protein Synthesis Inhibition

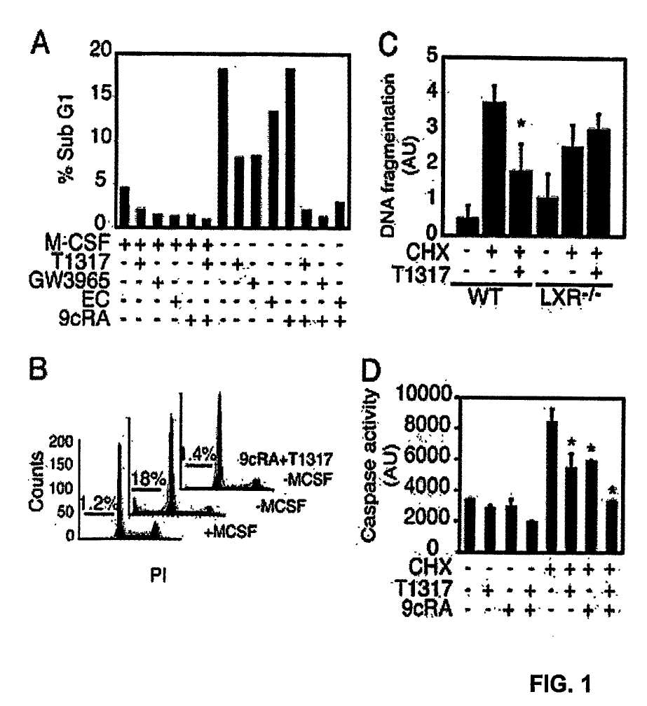

This example details the demonstration that LXR activation inhibits macrophage apoptosis. The inventor's discovered that treatment of bone marrow-derived macrophages (BMDMs) with LXR agonists improved their survival in the setting of growth factor withdrawal. Therefore the inventor's investigated potential roles of LXRs in regulation of macrophage apoptosis. Culturing BMDMs for 36 h in the absence of their specific growth factor (macrophage-colony stimulating factor, M-CSF) resulted in increased levels of cells with sub-G1 DNA content, an indicator of apoptosis-induced DNA fragmentation (FIG. 1a,b). This process was attenuated when macrophages were pre-incubated with the synthetic LXR agonists T1317 or GW3965 or the natural agonist 24(S), 25-epoxycholesterol (EC). 9 cis-retinoid acid (9cRA), a ligand for the RXR heterodimeric partner of LXRs, had little effect on sub-G1 content, but markedly enhanced the effects of three LXR-specific agonists (FIG. 1a,b).

The inventor's extended these studies to other modes of macrophage apoptosis in order to assess whether the protective effects of LXRs are limited to the control of programmed cell death caused by growth factor withdrawal. As a strategy to subvert normal host defense responses, a number of pathogens are armed with virulence factors that lead to rapid death of host macrophages (Weinrauch et al. (1999) Annu Rev Microbiol 53, 155-87). These virulence determinants include pore-forming toxins, protein synthesis inhibitors, superantigens and inhibitors of pro-survival signaling. In particular, macrophages are very sensitive to protein synthesis inhibition (Yang et al. (2000) Toxicol Appl Pharmacol 164, 149-60; Hsu et al. (2004) Nature 428, 341-5). Consistent with this, treatment of macrophages with cycloheximide (CHX) resulted in increased DNA fragmentation (FIG. 1c) and caspase activation (FIG. 1d). Preincubating the cells with T1317 for 24 h attenuated the apoptotic process induced by CHX in wild type macrophages, but not in LXR-deficient macrophages (FIG. 1c). Combined treatment of macrophages with synthetic or natural LXR agonists and 9cRA resulted in an additive inhibition of caspase activation (FIG. 1d). Similar results were obtained when the macrophage apoptotic program was stimulated by anisomycin (Streptomyces griseolus).

Macrophages were prestimulated with the indicated combinations of LXR and RXR agonists for 18 h and then deprived of M-CSF for 24 h. Ligands were replaced during the deprivation phase. The percentage of fragmented DNA (subGl population) is indicated in the graphic (PI, propidium iodide) as shown in FIG. 1a and 1b). WT and LXR^{-/-} macrophages (lacking both LXRα and LXRP) were plated at subconfluent densities, treated with vehicle or T1317, and then incubated with cycloheximide (CHX, 10 μg/ml) for 6 h. Macrophage apoptosis was determined by DNA fragmentation as shown in FIG. 1c. * p<0.05 vs treatment with CHX alone. Macrophages (40,000 cells/well) were pre-stimulated with vehicle, T1317 (1 μM), 9cis-retinoic acid (9cRA) (1 μM) or a combination of both for 24 h and then treated with CHX (10 μg/ml) for 5 h. General caspase activity was measured by fluorimetry as shown in FIG. 1d. Error bars represent standard deviations. * p<0.05 vs treatment with CHX alone.

### Example 3

### LXR And RXR Activation Protects Macrophages from Pathogen-Induced Apoptosis

This example details the demonstration that LXR and RXR promote macrophage survival in the face of bacterial infection. Recent studies have identified the p38MAPK (p38 mitogen-activated protein kinase) pathway as a target for the action of lethal factor, a virulence determinant from Bacillus anthracis (Park et al. (2002) Science 297, 2048-51). Inhibition of the p38MAPK cascade sensitizes macrophages to programmed cell death in response to activation of TLR4 (Hsu et al. (2004) Nature 428, 341-5; Park et al. (2002) Science 297, 2048-51). Treatment of BMDMs with LXR and RXR agonists resulted in decreased levels of annexin V staining after the combined incubation with LPS and the p38 inhibitor SB202190 (FIG. 2a,b). The inventor's evaluated the possibility that LXR and RXR agonists could protect macrophages from apoptosis due to infection with B. anthracis and other bacterial pathogens. Indeed, preincubation with a combination of LXR and RXR agonists significantly reduced the apoptotic responses, measured by TUNEL staining, that were elicited by infection with B. anthracis, E. coli, and the S. typhimurium strain SL1344/SipB⁻ (FIG. 2c). Although in some cases the antiapoptotic effects of LXR/RXR agonists could be overcome at high multiplicities of infection, these findings suggest that LXR and RXR promote macrophage survival in the face of bacterial infection.

Specifically, as shown in FIG. 2a, LXR and RXR activation protects macrophages from apoptosis induced by the combination of LPS and the p38 inhibitor SB202190 as determined by the percentage of annexin V-positive cells. Representative photomicrographs of each treatment [SBL, SB202190 (5 μM)+LPS (100 ng/ml); 9cT, 9cRA (1 μM)+T1317 (1 μM)] are shown in FIG. 2b. FIG. 2c depicts the effect of a combination of T1317 and 9cRA on apoptotic responses of macrophages exposed to the indicated multiplicity of infections (MOIs) of B. anthracis, E. coli, and S. typhimurium SL1344/SipB⁻. Error bars represent standard deviations. * p<0.05 vs bacterial exposure in the absence of ligands.

### Example 4

### Time Requirements for Effects of LXR/RXR Agonists on Macrophage Survival and Identification of Candidate Genes

This example details the demonstration that LXR and RXR regulate expression of pro- and anti-apoptotic factors. In order to characterize mechanisms of LXR-mediated protection from apoptosis, macrophages were preincubated with agonists at different time points before addition of the pro-apoptotic signal. Interestingly, inhibition of apoptosis in response to either anisomycin or the combination of SB202190 and LPS took place after a 12 h preincubation of the cells with LXR and RXR ligands (FIG. 3a,b). To identify ligand-regulated anti-apoptotic genes, expression-profiling experiments were performed using Affymetrix U74A and Codelink Uniset Mouse 1 microarrays. Because the anti-apoptotic effects of LXR agonists were strongly potentiated by 9cRA, microarray experiments examined effects of the LXR agonist T1317 alone and in combination with 9cRA. While T1317 alone had relatively modest effects on expression of genes with functional annotations linked to apoptosis, the combination of T1317 and 9cRA strongly regulated several pro- and anti-apoptotic genes (FIG. 3c). The most significantly upregulated gene with a functional annotation linked to inhibition of apoptosis was AIM4, also known as CT-2/Api6 (Haruta et al. (2001) J Biol Chem 276, 22910-4; Miyazaki et al. (1999) J Exp Med 189, 413-22). AlN4 was recently demonstrated to be induced by LXR agonists in liver (Maxwell et al., (2003) J Lipid Res 44, 2109-19). In addition, the anti-apoptotic regulators Birc1a (also known as Neuro AIP1) and Bcl-X_{L} were upregulated 3.3-fold and 2.9-fold, respectively (FIG. 3c). The combination of T1317 and 9cRA also significantly downregulated the proapoptotic regulators/effectors: Dnase1L3 (DNase γ), Caspases 1, 4/11, 7 and 12, Fas ligand, Cidea, and peptidoglycan recognition protein Tag7. These results were confirmed in two independent microarray experiments using Codelink Mouse Uniset I microarrays, and were validated for the overlapping sets of genes using Affymetrix U74A microarrays. In concert, these findings indicate that the combination of LXR and RXR agonists exert anti-apoptotic effects by coordinately regulating several pro- and anti-apoptotic genes.

Macrophages were preincubated with T1317 and 9cRA for specified times and then stimulated with anisomycin (Aniso) or SB202190+LPS for 6 h. The levels of caspase activity or % annexin V-positive cells at each time point are shown in FIGS. 3a and 3b, respectively. Error bars represent standard deviations. * p<0.05 vs treatment with anisomycin (a) or SB+LPS (b) alone. mRNA samples from macrophages stimulated with vehicle or the combination of T1317 (1 μM) and 9cRA (1 μM) for 16 h were subjected to expression profile analysis using Codelink Mouse Uniset 1 microarrays. The relative expression levels of genes with annotations linked to apoptosis changing by a factor of at least 1.5-fold are illustrated in FIG. 3c. Values are means of biological replicates. Changes in gene expression for AIM, Birc1a, Bcl-xL, Dnase1L3, Caspases 1, 7, 11 and 12 were independently confirmed by Northern blot analysis.

### Example 5

### Activation of LXR Antagonizes the Pro-Apoptotic Program Induced by Engagement of RXR

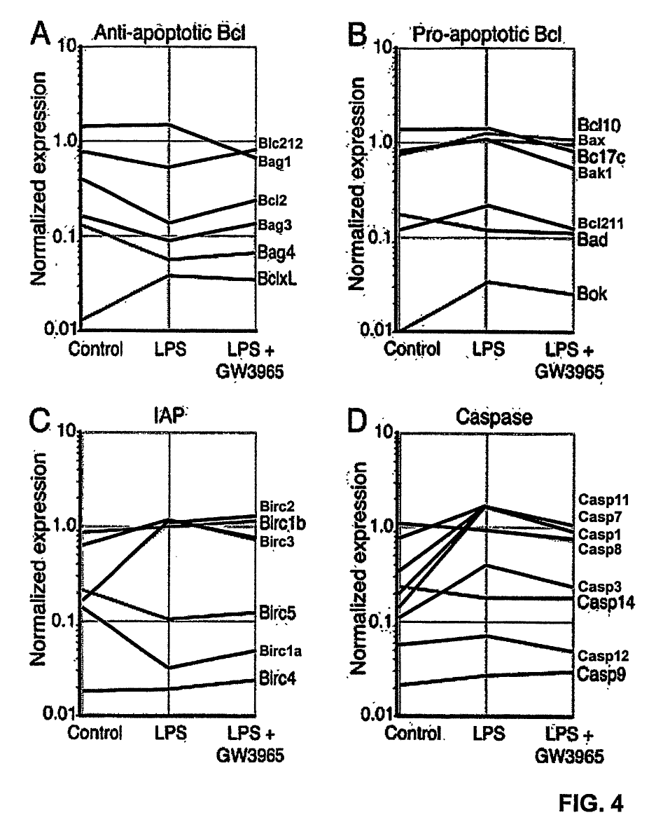

This example details the demonstration that LXRs inhibit apoptosis by coordinately regulating a network of genes that control programmed cell death. An additional series of microarray experiments was performed to evaluate the influence of LXR activation on regulation of the apoptotic program induced by engagement of TLR4 (FIG. 4). Macrophages were incubated with GW3965 or vehicle for 16 h and then treated with LPS for 6 hours. Of 86 genes with functional annotations linked to apoptosis and expressed in at least one condition, 23 genes were altered more than 1.5-fold by LPS treatment. Categorizing these genes into pro- and anti-apoptotic functions indicated that the overall response to TLR4 engagement was primarily pro-apoptotic, illustrated for selected categories of genes in FIG. 4. The dominant effect of LXR activation was to counter-regulate a subset of the pro-apoptotic program of gene expression induced by LPS. For example, the LXR agonist attenuated LPS-dependent downregulation of the anti-apoptotic proteins Bcl2, Bag3 and Birc1a. Conversely, LXR activation inhibited LPS-dependent induction of the pro-apoptotic factors Bax, Bak, Bcl211, and caspases 1, 3, 4/11, 7, 8 and 12. Together, these findings provided another independent line of evidence indicating that LXRs inhibit apoptosis by coordinately regulating a network of genes that control programmed cell death.

Macrophages were incubated with the LXR agonist GW3965 (1 μM) or vehicle for 16 h prior to treatment with LPS (100 ng/ml) for 6 h. Total RNA was subjected to microarray analysis using Codelink Mouse Uniset 1 microarrays. Relative expression levels for selected categories of pro-apoptotic and anti-apoptotic genes are illustrated. Genes exhibiting a response to GW3965 predicted to be pro-apoptotic include Bagl and Birc3. Genes exhibiting a response predicted to be anti-apoptotic Include Bcl212, Bcl2, Bag3, Bax, Bak1, Bcl211, Birc2, Birc1a, and Caspases 1, 3, 7, 8, 11 and 12. FIG. 4a) Anti-apoptotic members of the Bag and Bcl families. FIG. 4b) Pro-apoptotic members of the Bcl family. FIG. 4c) Members of the anti-apoptotic baculovirus IAP repeat-containing (Birc) family. FIG. 4d) Members of the caspase family.

### Example 6

### AIM is Synergistically Induced by LXR and RXR Agonists and Contributes to Their Anti-Apoptotic Effects

This example details the demonstration that induction of AIM expression contributes to the mechanism by which LXR and RXR agonists protect against apoptosis. Nonetheless, knowledge of the mechanism is not required to make and use the invention. AIM expression was initially evaluated in differentiated macrophages treated with LXR agonists. AIM mRNA levels were highly induced at 12 to 24 h of stimulation with T1317, which is somewhat delayed in comparison to ABCA1 (ATP-binding cassette, sub-family A (ABC1), member 1) and other direct LXR target genes (FIG. 5a). The combination of T1317 and 9cRA led to a much stronger induction of AIM, with maximal levels of expression again occurring at 24 h, consistent with the results of microarray experiments. Both the time course of AIM induction and synergistic effects of T1317 and 9cRA correlated with the time course requirements and combinatorial effects of both ligands on inhibition of apoptosis shown in FIG. 3. Compared to wild type macrophages (FIG. 5a), significantly lower amounts of AIM were induced in LXR deficient macrophages (FIG. 5b). AIM expression could also be induced by EC, indicating that it is subject to regulation by natural LXR ligands (FIG. 5c). Several combinations of the AIM promoter and upstream or downstream genomic elements that were found to be insufficient to drive LXR-dependent reporter gene expression in macrophage cell lines, raising the possibility that it is an indirect target of LXR/RXR heterodimers. Nonetheless, knowledge of the mechanism is not required to make and use the invention.

To investigate whether AIM induction contributes to the anti-apoptotic effects of LXR agonists, the inventors inhibited its expression using AIM-specific siRNAs. Primary macrophages were transfected with either siRNAs directed against AIM (SEQ ID NOs: 1 and 2), or a control siRNA (SEQ ID NO:3) designed to be unable to direct degradation of any known mouse gene. The cells were then stimulated with 9cRA and T1317 and expression of AIM was determined 24 h later by Northern blotting. As illustrated in FIG. 5d, transfection of macrophages with the siRNA directed against AIM reduced AIM mRNA expression by approximately 75%. Under these conditions, siRNA against AIM partially inhibited the ability of LXR and RXR agonists to protect macrophages from anisomycin-induced apoptosis (FIG. 5e). In contrast, LXR and RXR agonists were fully capable of inhibiting anisomycin-induced apoptosis in macrophages transfected with the control siRNA. These results indicate that induction of AIM expression contributes to the mechanism by which LXR and RXR agonists protect against apoptosis.

Specifically, macrophages were stimulated with T1317 or the combination of T1317 and 9cRA for the indicated times (FIG. 5a). Wild type and LXR^{-/-} macrophages were incubated for 24 h with T1317, 9cRA or a combination of both (FIG. 5b). Expression of AIM and other LXR target genes was analyzed by Northern blotting. As shown in FIG. 5c AIM is induced by 24(S),25-epoxycholesterol (EC) (10 μM). FIG. 5d illustrates that transfection of bone marrow-derived macrophages with a siRNA against AIM, significantly reduces AIM RNA levels, while FIG. 5e illustrates that reduction of AIM expression reduces anti-apoptotic activities of LXR and RXR agonists. Macrophages were transfected with a control siRNA, or a siRNA directed against AIM. The cells were then stimulated for 24 h with T1317, 9cRA or a combination of both, and then treated with anisomycin for 5 hours. Relative caspase activity was measured as an indicator of apoptosis. Each treatment was performed in triplicate. Error bars represent standard deviations. * p=0.045 vs anisomycin treatment alone. ** p=0.011 vs anisomycin alone. * p=0.055 vs anisomycin alone.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for modulating apoptosis, comprising administering an agent to a cell, wherein the cell comprises a liver X receptor (LXR) and wherein said administering increases activity of said LXR thereby modulating apoptosis.

2. The method of claim 1, wherein said modulating comprises reducing apoptosis.

3. The method of claim 1, wherein said agent comprises one or more of a small molecule, a protein, a peptide, a peptidomimetic, and a nucleic acid.

4. The method of claim 1, wherein said agent is an LXR agonist comprising one or more of a 24(S),25-epoxycholesterol (EC), T1317, and GW3965.

5. The method of claim 1, wherein said agent comprises an LXR agonist and a retinoid x receptor (RXR) agonist.

6. The method of claim 1, wherein said cell is a myeloid cell.

7. The method of claim 6, wherein said myeloid cell is a macrophage.

8. A method of treating a microbial infection of a cell, comprising:
a) providing:
i) a cell with one or more symptoms of a microbial infection, wherein said cell comprises one or both of a liver X receptor (LXR) and a retinoid X receptor (RXR); and
ii) a composition comprising an agent, wherein said agent comprises one or both of a LXR agonist and a RXR agonist; and
b) contacting said cell with said composition under conditions suitable for increasing activity of one or both of LXR and RXR such that the one or more symptoms of said microbial infection are reduced.

9. The method of claim 8, wherein said cell is in a population of cells, a tissue or an animal.

10. The method of claim 9, wherein said animal is a human or other mammal.

11. The method of claim 8, wherein said microbial infection comprises a bacterial infection.

12. The method of claim 11, wherein said bacterial infection comprises an infection with bacteria selected from the group consisting of Bacillus species, Escherichia species, Salmonella species, Shigella species, Yersinia species, Listeria species, Legionella species, Mycobacterium species, Streptococcus species and Haemophilus species.

13. The method of claim 8, wherein said agent comprises one or more of a 24(S),25-epoxycholesterol (EC), T1317, GW3965, and 9-cis-retinoic acid (9cRA).

14. The method of claim 8, wherein said cell is a myeloid cell.

15. The method of claim 14, wherein said myeloid cell is a macrophage.

16. The method of claim 8, wherein said one or more symptoms of said microbial infection comprise microbe-induced apoptosis.

17. A method of treating microbial infection of a cell, comprising:
a) providing:
i) a cell suspected of having a microbial infection, wherein said cell comprises an anti-apoptotic gene; and
ii) a composition comprising an agent for increasing activity of said anti-apoptotic gene; and
b) contacting said cell with said composition under conditions such that expression of said anti-apoptotic gene of said cell is increased.

18. The method of claim 17, wherein said cell is in a population of cells, a tissue or an animal.

19. The method of claim 18, wherein said animal is a human or other mammal.

20. The method of claim 17, wherein said microbial infection comprises a bacterial infection.

21. The method of claim 20, wherein said bacterial infection comprises an infection with bacteria selected from the group consisting of Bacillus species, Escherichia species, Salmonella species, Shigella species, Yersinia species, Listeria species, Legionella species, Mycobacterium species, Streptococcus species and Haemophilus species.

22. The method of claim 17, wherein said agent comprises one or more of a 24(S),25-epoxycholesterol (EC), T1317, GW3965, and 9-cis-retinoic acid (9cRA).

23. The method of claim 17, wherein said cell is a myeloid cell.

24. The method of claim 23, wherein said myeloid cell is a macrophage.

25. The method of claim 17, wherein said anti-apoptotic gene comprises one or more AIM, Birc1a, and Bcl-X_{L}.

26. A method for treating microbial infection of a cell, comprising:
a) providing:
i) a cell suspected of having a microbial infection, wherein said cell comprises a pro-apoptotic gene; and
ii) a composition comprising an agent for decreasing activity of said pro-apoptotic gene; and
b) contacting said cell with said composition under conditions such that expression of said pro-apoptotic gene of said cell is decreased.

27. The method of claim 26, wherein said cell is in a population of cells, a tissue or an animal.

28. The method of claim 27, wherein said animal is a human or other mammal.

29. The method of claim 26, wherein said microbial infection comprises a bacterial infection.

30. The method of claim 29, wherein said bacterial infection comprises an infection with bacteria selected from the group consisting of Bacillus species, Escherichia species, Salmonella species, Shigella species, Yersinia species, Listeria species, Legionella species, Mycobacterium species, Streptococcus species and Haemophilus species.

31. The method of claim 26, wherein said agent comprises one or more of a 24(S),25-epoxycholesterol (EC), T1317, GW3965, and 9-cis-retinoic acid (9cRA).

32. The method of claim 26, wherein said cell is a myeloid cell.

33. The method of claim 32, wherein said myeloid cell is a macrophage.

34. The method of claim 26, wherein said pro-apoptotic gene comprises one or more deoxyribonuclease I-like 3 (Dnase1L3), Caspase 1, Caspase 4, Caspase 7, Caspase 11, Caspase 12, Fas ligand, cell death-inducing DFFA-like effector A (CIDE-A), and peptidoglycan recognition protein (Tag7).
